SEQUENCE LISTING (18 sequences)

SEQ ID NO: 1 - protein, 189 aa, Artificial Sequence
  Erv1 protein
MKAIDKMTDNPPQEGLSGRKIIYDEDGKPCRSCNTLLDFQYVTGKISNGLKNLSSNGKLAGTGALTGEASELMPGSRTYRKVDPPDVEQLGRSSWTLLHSVAASYPAQPTDQQKGEMKQFLNIFSHIYPCNWCAKDFEKYIRENAPQVESREELGRWMCEAHNKVNKKLRKPKFDCNFWEKRWKDGWDE

SEQ ID NO: 2 - protein, 563 aa, Artificial Sequence
  Ero1 protein
MRLRTAIATLCLTAFTSATSNNSYIATDQTQNAFNDTHFCKVDRNDHVSPSCNVTFNELNAINENIRDDLSALLKSDFFKYFRLDLYKQCSFWDANDGLCLNRACSVDVVEDWDTLPEYWQPEILGSFNNDTMKEADDSDDECKFLDQLCQTSKKPVDIEDTINYCDVNDFNGKNAVLIDLTANPERFTGYGGKQAGQIWSTIYQDNCFTIGETGESLAKDAFYRLVSGFHASIGTHLSKEYLNTKTGKWEPNLDLFMARIGNFPDRVTNMYFNYAVVAKALWKIQPYLPEFSFCDLVNKEIKNKMDNVISQLDTKIFNEDLVFANDLSLTLKDEFRSRFKNVTKIMDCVQCDRCRLWGKIQTTGYATALKILFEINDADEFTKQHIVGKLTKYELIALLQTFGRLSESIESVNMFEKMYGKRLNGSENRLSSFFQNNFFNILKEAGKSIRYTIENINSTKEGKKKTNNSQSHVFDDLKMPKAEIVPRPSNGTVNKWKKAWNTEVNNVLEAFRFIYRSYLDLPRNIWELSLMKVYKFWNKFIGVADYVSEETREPISYKLDIQ

SEQ ID NO: 3 - DNA, 570 bp, Artificial Sequence
  ERV1
atgaaagcaatagataaaatgacggataatccaccacaagaaggcttaagtgggaggaaaataatatatgacgaagatggcaaaccttgccgatcatgtaacaccctacttgactttcagtacgtgaccgggaagatatctaatggcctgaagaacctctcatctaacggtaaactagcaggtacgggggctctcactggcgaagcttcagagttgatgcctggctcaagaacatacaggaaggttgaccctcctgacgtagagcaactaggtagatcttcatggacgctgttacactctgtagctgccagctatcctgctcaacctacagaccaacagaagggtgaaatgaaacagttcttgaatatcttctcacatatttatccttgcaactggtgtgctaaagactttgaaaaatatatcagagaaaatgcaccacaagttgagtcaagagaagaacttgggaggtggatgtgtgaagcccacaataaagtcaataagaaattgaggaagcccaaatttgactgtaatttctgggaaaaaagatggaaggacggctgggacgaataa

SEQ ID NO: 4 - DNA, 1692 bp, Artificial Sequence
  ERO1
atgagattaagaaccgccattgccacactgtgcctcacggcttttacatctgcaacttcaaacaatagctacatcgccaccgaccaaacacaaaatgcctttaatgacactcacttttgtaaggtcgacaggaatgatcacgttagtcccagttgtaacgtaacattcaatgaattaaatgccataaatgaaaacattagagatgatctttcggcgttattaaaatctgatttcttcaaatactttcggctggatttatacaagcaatgttcattttgggacgccaacgatggtctgtgcttaaaccgcgcttgctctgttgatgtcgtagaggactgggatacactgcctgagtactggcagcctgagatcttgggtagtttcaataatgatacaatgaaggaagcggatgatagcgatgacgaatgtaagttcttagatcaactatgtcaaaccagtaaaaaacctgtagatatcgaagacaccatcaactactgtgatgtaaatgactttaacggtaaaaacgccgttctgattgatttaacagcaaatccggaacgatttacaggttatggtggtaagcaagctggtcaaatttggtctactatctaccaagacaactgttttacaattggcgaaactggtgaatcattggccaaagatgcattttatagacttgtatccggtttccatgcctctatcggtactcacttatcaaaggaatatttgaacacgaaaactggtaaatgggagcccaatctggatttgtttatggcaagaatcgggaactttcctgatagagtgacaaacatgtatttcaattatgctgttgtagctaaggctctctggaaaattcaaccatatttaccagaattttcattctgtgatctagtcaataaagaaatcaaaaacaaaatggataacgttatttcccagctggacacaaaaatttttaacgaagacttagtttttgccaacgacctaagtttgactttgaaggacgaattcagatctcgcttcaagaatgtcacgaagattatggattgtgtgcaatgtgatagatgtagattgtggggcaaaattcaaactaccggttacgcaactgccttgaaaattttgtttgaaatcaacgacgctgatgaattcaccaaacaacatattgttggtaagttaaccaaatatgagttgattgcactattacagactttcggtagattatctgaatctattgaatctgttaacatgttcgaaaaaatgtacgggaaaaggttaaacggttctgaaaacaggttaagctcattcttccaaaataacttcttcaacattttgaaggaggcaggcaaatcgattcgttacaccatagagaacatcaattccactaaagaaggaaagaaaaagactaacaattctcaatcacatgtatttgatgatttaaaaatgcccaaagcagaaatagttccaaggccctctaacggtacagtaaataaatggaagaaagcttggaatactgaagttaacaacgttttagaagcattcagatttatttatagaagctatttggatttacccaggaacatctgggaattatctttgatgaaggtatacaaattttggaataaattcatcggtgttgctgattacgttagtgaggagacacgagagcctatttcctataagctagatatacaataa

SEQ ID NO: 5 - protein, 483 aa, Artificial Sequence
  Glr1 protein
MLSATKQTFRSLQIRTMSTNTKHYDYLVIGGGSGGVASARRAASYGAKTLLVEAKALGGTCVNVGCVPKKVMWYASDLATRVSHANEYGLYQNLPLDKEHLTFNWPEFKQKRDAYVHRLNGIYQKNLEKEKVDVVFGWARFNKDGNVEVQKRDNTTEVYSANHILVATGGKAIFPENIPGFELGTDSDGFFRLEEQPKKVVVVGAGYIGIELAGVFHGLGSETHLVIRGETVLRKFDECIQNTITDHYVKEGINVHKLSKIVKVEKNVETDKLKIHMNDSKSIDDVDELIWTIGRKSHLGMGSENVGIKLNSHDQIIADEYQNTNVPNIYSLGDVVGKVELTPVAIAAGRKLSNRLFGPEKFRNDKLDYENVPSVIFSHPEAGSIGISEKEAIEKYGKENIKVYNSKFTAMYYAMLSEKSPTRYKIVCAGPNEKVVGLHIVGDSSAEILQGFGVAIKMGATKADFDNCVAIHPTSAEELVTMR

SEQ ID NO: 6 - DNA, 1452 bp, Artificial Sequence
  GLR1
atgctttctgcaaccaaacaaacatttagaagtctacagataagaactatgtccacgaacaccaagcattacgattacctcgtcatcgggggtggctcagggggtgttgcttccgcaagaagagctgcatcttatggtgcgaagacattactagttgaagctaaggctcttggtggtacctgtgttaacgtgggttgtgttccgaagaaagtcatgtggtatgcttctgacctcgctactagagtatcccatgcaaatgaatatggattatatcagaatcttccattagataaagagcatttgacttttaattggccagaatttaagcagaaaagggatgcttatgtccataggttgaacggtatataccagaagaatttagaaaaagaaaaagtggatgttgtatttggatgggctagattcaataaggacggtaatgttgaagttcagaaaagggataatactactgaagtttactccgctaaccatattttagttgcgaccggtggaaaggctattttccccgaaaacattccaggtttcgaattaggtactgattctgatgggttctttagattggaagaacaacctaagaaagttgttgttgttggcgctggttatattggtattgagctagcaggtgtgttccatgggctgggatccgaaacgcacttggtaattagaggtgaaactgtcttgagaaaatttgatgaatgcatccagaacactattactgaccattacgtaaaggaaggcatcaacgttcataaactatccaaaattgttaaggtggagaaaaatgtagaaactgacaaactgaaaatacatatgaatgactcaaagtccatcgatgacgttgacgaattaatttggacaattggacgtaaatcccatctaggtatgggttcagaaaatgtaggtataaagctgaactctcatgaccaaataattgctgatgaatatcagaacaccaatgttccaaacatttattctctaggtgacgttgttggaaaagttgaattgacacctgtcgctattgcagcgggcagaaagctgtctaatagactgtttggtccagagaaattccgtaatgacaaactagattacgagaacgtccccagcgtaattttctcacatcctgaagccggttccattggtatttctgagaaggaagccattgaaaagtacggtaaggagaatataaaggtctacaattccaaatttaccgccatgtactatgctatgttgagtgagaaatcacccacaagatataaaattgtttgtgcgggaccaaatgaaaaggttgtcggtctgcacattgttggtgattcctctgcagaaatcttgcaagggttcggtgttgctataaagatgggtgccactaaggctgatttcgataattgtgttgctattcatccgactagcgcagaagaattggttactatgagatga

SEQ ID NO: 7 - protein, 678 aa, Artificial Sequence
  Gsh1p
MGLLALGTPLQWFESRTYNEHIRDEGIEQLLYIFQAAGKRDNDPLFWGDELEYMVVDFDDKERNSMLDVCHDKILTELNMEDSSLCEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 8 - DNA, 2037 bp, Artificial Sequence
  GSH1
atgggactcttagctttgggcacgcctttgcagtggtttgagtctaggacgtacaatgaacacataagggatgaaggtatcgagcagttgttgtatattttccaagctgctggtaaaagagacaatgaccctcttttttggggagacgagcttgagtacatggttgtagattttgatgataaggagagaaattctatgctcgacgtttgccatgacaagatactcactgagcttaatatggaggattcgtccctttgtgaggctaacgatgtgagttttcaccctgagtatggccggtatatgttagaggcaacaccagcttctccatatttgaattacgtgggtagttacgttgaggttaacatgcaaaaaagacgtgccattgcagaatataagctatctgaatatgcgagacaagatagtaaaaataacttgcatgtgggctccaggtctgtccctttgacgctgactgtcttcccgaggatgggatgccccgactttattaacattaaggatccgtggaatcataaaaatgccgcttccaggtctctgtttttacccgatgaagtcattaacagacatgtcaggtttcctaacttgacagcatccatcaggaccaggcgtggtgaaaaagtttgcatgaatgttcccatgtataaagatatagctactccagaaacggatgactccatctacgatcgagattggtttttaccagaagacaaagaggcgaaactggcttccaaaccgggtttcatttatatggattccatgggttttggcatgggctgttcgtgcttacaagtgacctttcaggcacccaatatcaacaaggcacgttacctgtacgatgcattagtgaattttgcacctataatgctagccttctctgccgctgcgcctgcttttaaaggttggctagccgaccaagatgttcgttggaatgtgatatctggtgcggtggacgaccgtactccgaaggaaagaggtgttgcgccattactacccaaatacaacaagaacggatttggaggcattgccaaagacgtacaagataaagtccttgaaataccaaagtcaagatatagttcggttgatcttttcttgggtgggtcgaaatttttcaataggacttataacgacacaaatgtacctattaatgaaaaagtattaggacgactactagagaatgataaggcgccactggactatgatcttgctaaacattttgcgcatctctacataagagatccagtatctacattcgaagaactgttgaatcaggacaacaaaacgtcttcaaatcactttgaaaacatccaaagtacaaattggcagacattacgttttaaaccccccacacaacaagcaaccccggacaaaaaggattctcctggttggagagtggaattcagaccatttgaagtgcaactattagattttgagaacgctgcgtattccgtgctcatatacttgattgtcgatagcattttgaccttttccgataatattaacgcatatattcatatgtccaaagtatgggaaaatatgaagatagcccatcacagagatgctatcctatttgaaaaatttcattggaaaaaatcatttcgcaacgacaccgatgtggaaactgaagattattctataagcgagattttccataatccagagaatggtatatttcctcaatttgttacgccaatcctatgccaaaaagggtttgtaaccaaagattggaaagaattaaagcattcttccaaacacgagagactatactattatttaaagctaatttctgatagagcaagcggtgaattgccaacaacagcaaaattctttagaaattttgtactacaacatccagattacaaacatgattcaaaaatttcaaagtcgatcaattatgatttgctttctacgtgtgatagacttacccatttagacgattcaaaaggtgaattgacatcctttttaggagctgaaattgcagaatatgtaaaaaaaaataagccttcaatagaaagcaaatgttaa

SEQ ID NO: 9 - protein, 491 aa, Artificial Sequence
  Gsh2p
MAHYPPSKDQLNELIQEVNQWAITNGLSMYPPKFEENPSNASVSPVTIYPTPIPRKCFDEAVQIQPVFNELYARITQDMAQPDSYLHKTTEALALSDSEFTGKLWSLYLATLKSAQYKKQNFRLGIFRSDYLIDKKKGTEQIKQVEFNTVSVSFAGLSEKVDRLHSYLNRANKYDPKGPIYNDQNMVISDSGYLLSKALAKAVESYKSQQSSSTTSDPIVAFIVQRNERNVFDQKVLELNLLEKFGTKSVRLTFDDVNDKLFIDDKTGKLFIRDTEQEIAVVYYRTGYTTTDYTSEKDWEARLFLEKSFAIKAPDLLTQLSGSKKIQQLLTDEGVLGKYISDAEKKSSLLKTFVKIYPLDDTKLGREGKRLALSEPSKYVLKPQREGGGNNVYKENIPNFLKGIEERHWDAYILMELIEPELNENNIILRDNKSYNEPIISELGIYGCVLFNDEQVLSNEFSGSLLRSKFNTSNEGGVAAGFGCLDSIILY

SEQ ID NO: 10 - DNA, 1476 bp, Artificial Sequence
  GSH2
atggcacactatccaccttccaaggatcaattgaatgaattgatccaggaagttaaccaatgggctatcactaatggattatccatgtatcctcctaaattcgaggagaacccatcaaatgcatcggtgtcaccagtaactatctatccaaccccaattcctaggaaatgttttgatgaggccgttcaaatacaaccggtattcaatgaattatacgcccgtattacccaagatatggcccaacctgattcttatttacataaaacaactgaagcgttagctctatcagattccgagtttactggaaaactgtggtctctataccttgctaccttaaaatctgcacagtacaaaaagcagaattttaggctaggtatatttagatcagattatttgattgataagaaaaagggtactgaacagattaagcaagtcgagtttaatacagtgtcagtgtcatttgcaggccttagcgagaaagttgatagattgcactcttatttaaatagggcaaacaagtacgatcctaaaggaccaatttataatgatcaaaatatggtcatttctgattcaggataccttttgtctaaggcattggccaaagctgtggaatcgtataagtcacaacaaagttcttctacaactagtgatcctattgtcgcattcattgtgcaaagaaacgagagaaatgtgtttgatcaaaaggtcttggaattgaatctgttggaaaaattcggtaccaaatctgttaggttgacgtttgatgatgttaacgataaattgttcattgatgataaaacgggaaagcttttcattagggacacagagcaggaaatagcggtggtttattacagaacgggttacacaaccactgattacacgtccgaaaaggactgggaggcaagactattcctcgaaaaaagtttcgcaataaaggccccagatttactcactcaattatctggctccaagaaaattcagcaattgttgacagatgagggcgtattaggtaaatacatctccgatgctgagaaaaagagtagtttgttaaaaacttttgtcaaaatatatcccttggatgatacgaagcttggcagggaaggcaagaggctggcattaagtgagccctctaaatacgtgttaaaaccacagcgggaaggtggcggaaacaatgtttataaagaaaatattcctaattttttgaaaggtatcgaagaacgtcactgggatgcatatattctcatggagttgattgaaccagagttgaatgaaaataatattatattacgtgataacaaatcttacaacgaaccaatcatcagtgaactaggaatttatggttgcgttctatttaacgacgagcaagttttatcgaacgaatttagtggctcattactaagatccaaatttaatacttcaaatgaaggtggagtggcggcaggattcggatgtttggacagtattattctttactag

SEQ ID NO: 11 - protein, 1515 aa, Artificial Sequence
  Ycf1p
MAGNLVSWACKLCRSPEGFGPISFYGDFTQCFIDGVILNLSAIFMITFGIRDLVNLCKKKHSGIKYRRNWIIVSRMALVLLEIAFVSLASLNISKEEAENFTIVSQYASTMLSLFVALALHWIEYDRSVVANTVLLFYWLFETFGNFAKLINILIRHTYEGIWYSGQTGFILTLFQVITCASILLLEALPKKPLMPHQHIHQTLTRRKPNPYDSANIFSRITFSWMSGLMKTGYEKYLVEADLYKLPRNFSSEELSQKLEKNWENELKQKSNPSLSWAICRTFGSKMLLAAFFKAIHDVLAFTQPQLLRILIKFVTDYNSERQDDHSSLQGFENNHPQKLPIVRGFLIAFAMFLVGFTQTSVLHQYFLNVFNTGMYIKSALTALIYQKSLVLSNEASGLSSTGDIVNLMSVDVQKLQDLTQWLNLIWSGPFQIIICLYSLYKLLGNSMWVGVIILVIMMPLNSFLMRIQKKLQKSQMKYKDERTRVISEILNNIKSLKLYAWEKPYREKLEEVRNNKELKNLTKLGCYMAVTSFQFNIVPFLVSCCTFAVFVYTEDRALTTDLVFPALTLFNLLSFPLMIIPMVLNSFIEASVSIGRLFTFFTNEELQPDSVQRLPKVKNIGDVAINIGDDATFLWQRKPEYKVALKNINFQAKKGNLTCIVGKVGSGKTALLSCMLGDLFRVKGFATVHGSVAYVSQVPWIMNGTVKENILFGHRYDAEFYEKTIKACALTIDLAILMDGDKTLVGEKGISLSGGQKARLSLARAVYARADTYLLDDPLAAVDEHVARHLIEHVLGPNGLLHTKTKVLATNKVSALSIADSIALLDNGEITQQGTYDEITKDADSPLWKLLNNYGKKNNGKSNEFGDSSESSVRESSIPVEGELEQLQKLNDLDFGNSDAISLRRASDATLGSIDFGDDENIAKREHREQGKVKWNIYLEYAKACNPKSVCVFILFIVISMFLSVMGNVWLKHWSEVNSRYGSNPNAARYLAIYFALGIGSALATLIQTIVLWVFCTIHASKYLHNLMTNSVLRAPMTFFETTPIGRILNRFSNDIYKVDALLGRTFSQFFVNAVKVTFTITVICATTWQFIFIIIPLSVFYIYYQQYYLRTSRELRRLDSITRSPIYSHFQETLGGLATVRGYSQQKRFSHINQCRIDNNMSAFYPSINANRWLAYRLELIGSIIILGAATLSVFRLKQGTLTAGMVGLSLSYALQITQTLNWIVRMTVEVETNIVSVERIKEYADLKSEAPLIVEGHRPPKEWPSQGDIKFNNYSTRYRPELDLVLKHINIHIKPNEKVGIVGRTGAGKSSLTLALFRMIEASEGNIVIDNIAINEIGLYDLRHKLSIIPQDSQVFEGTVRENIDPINQYTDEAIWRALELSHLKEHVLSMSNDGLDAQLTEGGGNLSVGQRQLLCLARAMLVPSKILVLDEATAAVDVETDKVVQETIRTAFKDRTILTIAHRLNTIMDSDRIIVLDNGKVAEFDSPGQLLSDNKSLFYSLCMEAGLVNEN

SEQ ID NO: 12 - DNA, 4548 bp, Artificial Sequence
  YCF1
atggctggtaatcttgtttcatgggcctgcaagctctgtagatctcctgaagggtttggacctatatccttttacggtgactttactcaatgcttcatcgacggtgtgatcctaaatctatcagcaattttcatgataaccttcggtatcagagatttagttaacctttgcaagaaaaaacactctggcatcaaatataggcggaattggattattgtctctaggatggcactagttctgttggagatagcgtttgtttcacttgcgtctttaaatatttctaaagaagaagcggaaaactttaccattgtaagtcaatatgcttctacaatgttatctttatttgttgctttagccttacactggatagaatacgatagatcagttgtagccaatacggtacttttattctattggctttttgaaacattcggtaattttgctaaactaataaatattctaattagacacacctacgaaggcatttggtattccggacaaacgggtttcatactaacgttattccaagtaataacatgtgccagtatcctgttacttgaagctcttccaaagaagccgctaatgccacatcaacacatacatcaaactttaacaagaagaaaaccaaatccatacgatagcgcaaacatattttccaggattaccttctcttggatgtcaggtttgatgaaaactggctatgaaaaatacttagtggaagcagatttatataaattaccgaggaactttagtagtgaagaactctctcaaaaattggagaaaaactgggaaaatgagttgaagcaaaaatcaaatccttcattatcatgggctatatgcagaacttttggatctaaaatgcttttagccgcattctttaaagcaattcatgatgttctagcatttactcaaccacaactactaaggattttaatcaagttcgtcacagactataacagtgagagacaggatgaccattcttctcttcaagggtttgaaaataaccacccacaaaaattacccattgtaagagggtttttgattgcgtttgctatgtttctggtgggctttactcagacatctgtcctgcatcaatatttcctgaatgtcttcaacacaggcatgtatattaagagcgccctaacggctttaatatatcaaaaatccttagtgctatctaatgaggcttctggactttcctctaccggtgacattgtcaatctcatgagtgtggatgttcaaaaattacaagatttaacacaatggctaaatttaatatggtcagggccttttcaaatcattatttgcttatattctctgtataagttgttgggaaattccatgtgggttggcgtgattatactagttattatgatgccattgaactcatttttgatgaggatacaaaagaagttgcaaaaatcccagatgaagtacaaagatgaaaggacccgtgttataagtgaaatactaaacaatattaaatctttgaagttatatgcatgggagaagccttatagggaaaagctagaagaagtaagaaataacaaagagttaaaaaatcttacaaaactaggatgttatatggctgtgacaagttttcagttcaatatagtaccattccttgtttcatgttgtacctttgctgtatttgtttatactgaggatagagcattgactactgacttagttttccctgctttgactctgttcaacctgctctcattcccactaatgattattcctatggttttaaattcttttatcgaagcttctgtttctattggtagattatttacattctttaccaatgaagagctacaaccagattcggttcagcgtttaccaaaagtaaaaaatattggcgatgtagccattaacattggagatgatgctacctttttatggcaacggaaaccggaatacaaagtagccttaaagaatattaatttccaagctaaaaaaggaaatttgacctgtattgttggtaaagttggcagtggtaaaacagctctattgtcatgcatgttaggtgatctattcagggttaaaggtttcgccaccgttcatggttctgttgcttatgtttcacaagttccatggataatgaatggtactgtaaaggaaaacattttatttgggcatagatacgacgcggaattttacgaaaaaacgatcaaggcctgtgcgttaactattgatcttgcaattttgatggatggagataagacattagttggcgagaaagggatctccttatctggaggacaaaaagctcgtttgtctttagcaagagcagtttatgcgagagctgacacttatttacttgatgatcctttggcagctgttgatgaacacgttgccaggcacttgatcgaacatgtgttgggtccaaatggtttattacatacaaaaacgaaggtattagccactaataaggtgagcgcgttatccatcgcagattctattgcattattagataatggagaaatcacacagcagggtacatatgatgagattacgaaggacgctgattcgccattatggaaattgctcaacaactatggtaaaaaaaataacggtaagtcgaatgaattcggtgactcctctgaaagctcagttcgagaaagtagtatacctgtagaaggagagctggaacaactgcagaaattaaatgatttggattttggcaactctgacgccataagtttaaggagggccagtgatgcaactttgggaagcatcgattttggtgacgatgaaaatattgctaaaagagagcatcgtgaacagggaaaagtaaagtggaacatttacctagagtacgctaaagcttgcaacccgaaaagcgtttgtgtattcatattgtttattgttatatcgatgttcctctctgttatgggtaacgtttggttgaaacattggtctgaagttaatagccgctatggatctaatccaaatgccgcgcgttacttggccatttattttgcacttggtattggttcagcactggcaacattaatccagacaatcgttctctgggttttttgtaccattcatgcctccaaatatttacacaacttgatgacaaactctgtgttgagagccccaatgacgttttttgaaacaacaccaatcggtagaattctaaacagattctcaaatgacatatacaaagtggatgctttattaggaagaacattttctcagtttttcgtcaatgcagtgaaagtcacattcactattacggttatctgtgcgacgacatggcaatttatcttcattatcattccactaagtgtgttttacatctactaccagcagtattacctgagaacatcaagggagttgcgtcgtttagactctattactaggtctccaatctactctcatttccaagagactttgggtggccttgcaacggttagaggttattctcaacagaaaaggttttcccacattaatcaatgccgcattgataataacatgagtgcgttctatccctctatcaatgctaaccgttggctagcatataggttggaacttattggttcaattatcattctaggtgctgcaactttatccgtttttagactaaaacaaggcacattaacggcaggtatggtgggtttatcattaagctatgctttacaaatcactcaaacgttaaattggattgttagaatgactgtggaagttgaaacgaatattgtttcagtggaaagaataaaggaatatgctgatttgaagagcgaggcacctttaatagttgaaggccacagaccacccaaagaatggccgagccagggtgatataaagtttaataattattccactcgttataggccggagcttgatcttgttctgaagcacattaatatacacattaaaccaaatgaaaaagttggtatcgtgggtagaacgggtgcgggaaaatcctcattaacgctagcattattcaggatgattgaggctagcgagggaaacatcgtaatcgacaacattgccatcaacgagattgggttatatgatttgagacataaattgtcaatcatacctcaggattctcaagtttttgagggcactgttcgtgagaacattgatcccattaaccaatacactgatgaagctatttggagggcattggaactttctcatttgaaagaacacgtgctatcaatgagcaatgacggattagatgcccaactaaccgaaggtggtggcaacttaagtgttggacaaagacaattattatgtcttgcaagagcaatgttggttccatcaaagattttggtgcttgatgaagccacggccgcagtcgacgtggagacagataaagtcgtccaagagacgattcgtactgctttcaaggacagaactatcttgaccatcgcgcatagactgaacacgataatggacagtgatagaatcatagtgttggacaatggtaaagtagccgagtttgactctccgggccagttattaagtgataacaaatcattgttctattcactgtgcatggaggctggtttggtcaatgaaaattaa

SEQ ID NO: 13 - DNA, 35 bp, Artificial Sequence
  ERV1-F
ggccgctagcatgaaagcaatagataaaatgacgg

SEQ ID NO: 14 - DNA, 33 bp, Artificial Sequence
  ERV1-R
ggccggatccttattcgtcccagccgtccttcc

SEQ ID NO: 15 - DNA, 36 bp, Artificial Sequence
  ERO1-F
ggccgctagcatgagattaagaaccgccattgccac

SEQ ID NO: 16 - DNA, 41 bp, Artificial Sequence
  ERO1-R
ggccggatccttattgtatatctagcttataggaaataggc

SEQ ID NO: 17 - DNA, 37 bp, Artificial Sequence
  YCF1-F
aaaaggatccatggctggtaatcttgtttcatgggcc

SEQ ID NO: 18 - DNA, 38 bp, Artificial Sequence
  YCF1-R
aaaactcgagttaattttcattgaccaaaccagcctcc

(19) United States
(12) Patent Application Publication
(10) Pub. No.: US 2018/0044710 A1
(43) Pub. Date: Feb. 15, 2018

(54) METHOD FOR PRODUCING GLUTATHIONE

(71) Applicants: KANEKA CORPORATION, Osaka-shi (JP); NATIONAL UNIVERSITY CORPORATION KOBE UNIVERSITY, Kobe-shi (JP)

(72) Inventors: Kiyotaka HARA, Kobe-shi (JP); Akihiko KONDO, Kobe-shi (JP); Akira IWASAKI, Takasago-shi (JP); Yuichi IWAMOTO, Takasago-shi (JP)

(73) Assignees: KANEKA CORPORATION, Osaka-shi (JP); NATIONAL UNIVERSITY CORPORATION KOBE UNIVERSITY, Kobe-shi (JP)

(21) Appl. No.: 15/555,392

(22) PCT Filed: Mar. 4, 2016
(86) PCT No.: PCT/JP2016/056845

§ 371 Date: Sep. 1, 2017

(30) Foreign Application Priority Data
Mar. 4, 2015 (JP) 2015-042909

(51) Int. Cl. C12P 21/02; C12N 9/00; C12N 15/81; C12N 9/02

(52) U.S. Cl. CPC C12P 21/02; C12N 9/0051; C12Y 108/03002; C12Y 108/01007; C12N 9/93; C12Y 603/02002; C12Y 603/02003; C12N 15/81

(57) ABSTRACT

The present invention aims to provide a yeast that is genetically modified so as to more highly produce glutathione, and a method of producing glutathione utilizing the yeast. The present invention provides a method of producing glutathione, including culturing a yeast whose thiol oxidase activity is increased as compared to the parent strain in a culture medium to produce glutathione, and recovering glutathione from the cultured broth obtained.

### TECHNICAL FIELD

The present invention relates to a yeast highly producing glutathione, which is used for pharmaceutical products, foods and the like, and a method of producing glutathione by using the yeast.

### BACKGROUND ART

Glutathione is known to exist as a reduced form or an oxidized form. The reduced glutathione is a peptide consisting of three amino acids, cysteine, glutamic acid and glycine. The oxidized glutathione is a compound in which the thiol groups of two reduced glutathione molecules form a disulfide bond. Glutathione exists in not only human body but also many living bodies such as other animals, plants, microorganisms and the like, and is an important compound for living bodies involved in scavenging action of reactive oxygens, detoxification, amino acid metabolisms and the like. As such, it has attracted attention in pharmaceutical, food and cosmetic industries. In addition, since it has been recently found that oxidized glutathione has effects such as promoting the growth of plants and the like, it has been expected to be used in various fields including agriculture.

Industrially, glutathione is currently produced by a fermentation method using yeast. Since glutathione is accumulated within yeast cells, in actual, it is a widely used method to culture yeasts and extract glutathione from the cultured cells. Accordingly, attempts to increase glutathione content in the cultured cells to improve glutathione producibility have been made, by inducing mutagenesis of yeast used for glutathione production or introducing an enzyme involved in glutathione synthesis using genetic recombination (patent documents 1, 2), or by adding L-glutamic acid, L-cysteine and glycine, which are three kinds of amino acids constituting glutathione, to a culture medium. In recent years, methods accumulating glutathione in oxidized form within yeast cells have also been reported. In non-patent document 1, a strain in which a glutathione reductase gene was destructed and the expression of a glutathione transport enzyme was enhanced resulted in increase of oxidized glutathione and 30% increase of total glutathione (oxidized+reduced glutathiones). Patent document 3 reports a method of increasing total glutathione amount including oxidized glutathione by the expression of glutathione peroxidase.

However, it is required to obtain a yeast more efficiently producing glutathione, in order to industrially produce glutathione at a lower cost.

### DOCUMENT LIST

### Patent Documents

Patent document 1: JP S64-51098 A
Patent document 2: JP 2012-213376 A
Patent document 3: JP 2014-64472 A

### Non-Patent Document

Non-patent document 1: Nature Chemical Biology 9, 119-125 (2013)

### SUMMARY OF THE INVENTION

### Problems to be Solved by the Invention

Under the above-mentioned background art, the present invention aims to provide a yeast that is genetically modified so as to more highly produce glutathione, and a method of producing glutathione utilizing the yeast.

### Means of Solving the Problems

To solve the aforementioned problem, the present inventors made intensive studies and found that an intracellular amount of glutathione including oxidized glutathione was increased in a yeast strain in which thiol oxidase activity was increased. In addition, when the activity of glutathione reductase, which is involved in reduction of oxidized glutathione, was reduced in the thiol oxidase activity-increased strain, surprisingly, a remarkable increase in glutathione production, namely a significant increase in intracellular glutathione amount (oxidized glutathione+reduced glutathione) was revealed, which resulted in the completion of the present invention.

Furthermore, it was confirmed that glutathione production can be more synergistically improved by increasing the activities of γ-glutamylcysteine synthetase (GSH1) and/or glutathione synthetase (GSH2) and/or glutathione transport enzyme (YCF1) in the thiol oxidase activity-increased strain.

Accordingly, the present invention relates to a method of producing glutathione characterized in culturing a yeast whose thiol oxidase activity is increased.

To be specific, the present invention provides the following.
[1] A method of producing glutathione, comprising culturing a yeast whose thiol oxidase activity is increased as compared to the parent strain in a culture medium to produce glutathione, and recovering glutathione from the cultured broth obtained.
[2] The production method according to the above-mentioned [1], wherein the thiol oxidase is selected from the following (a)-(f):
(a) a protein consisting of the amino acid sequence shown by SEQ ID NO: 1 or 2,
(b) a protein having the amino acid sequence shown by SEQ ID NO: 1 or 2, wherein 1 or plural amino acids are deleted, substituted, inserted and/or added, and having a thiol oxidase activity,
(c) a protein consisting of an amino acid sequence having 60% or more sequence identity to the amino acid sequence shown by SEQ ID NO: 1 or 2,
(d) a protein consisting of an amino acid sequence encoded by a nucleotide sequence shown by SEQ ID NO: 3 or 4,
(e) a protein consisting of an amino acid sequence encoded by a DNA that hybridizes with a DNA having a nucleotide sequence complementary to SEQ ID NO: 3 or 4 under stringent conditions, and,
(f) a protein consisting of an amino acid sequence encoded by a DNA having the nucleotide sequence shown by SEQ ID NO: 3 or 4 wherein 1 or plural nucleotides are substituted, deleted, inserted and/or added, and having a thiol oxidase activity.
[3] The production method according to the above-mentioned [1] or [2], wherein the yeast is a yeast whose glutathione reductase activity is reduced as compared to the parent strain.
[4] The production method according to the above-mentioned [3], wherein the glutathione reductase is selected from the following (a)-(f):
(a) a protein consisting of the amino acid sequence shown by SEQ ID NO: 5,
(b) a protein having the amino acid sequence shown by SEQ ID NO: 5, wherein 1 or plural amino acids are deleted, substituted, inserted and/or added, and having a glutathione reductase activity,
(c) a protein consisting of an amino acid sequence having 60% or more sequence identity to the amino acid sequence shown by SEQ ID NO: 5,
(d) a protein consisting of an amino acid sequence encoded by a nucleotide sequence shown by SEQ ID NO: 6,
(e) a protein consisting of an amino acid sequence encoded by a DNA that hybridizes with a DNA having a nucleotide sequence complementary to SEQ ID NO: 6 under stringent conditions, and,
(f) a protein consisting of an amino acid sequence encoded by a DNA having the nucleotide sequence shown by SEQ ID NO: 6 wherein 1 or plural nucleotides are substituted, deleted, inserted and/or added, and having a glutathione reductase activity.
[5] The production method according to any of the above-mentioned [1]-[4], wherein the yeast is a yeast in which the expression of γ-glutamylcysteine synthetase and/or glutathione synthetase is enhanced as compared to the parent strain.
[6] The production method according to the above-mentioned [5], wherein the γ-glutamylcysteine synthetase is selected from the following (a)-(f):
(a) a protein consisting of the amino acid sequence shown by SEQ ID NO: 7,
(b) a protein having the amino acid sequence shown by SEQ ID NO: 7, wherein 1 or plural amino acids are deleted, substituted, inserted and/or added, and having a γ-glutamylcysteine synthetase activity,
(c) a protein consisting of an amino acid sequence having 60% or more sequence identity to the amino acid sequence shown by SEQ ID NO: 7,
(d) a protein consisting of an amino acid sequence encoded by a nucleotide sequence shown by SEQ ID NO: 8,
(e) a protein consisting of an amino acid sequence encoded by a DNA that hybridizes with a DNA having a nucleotide sequence complementary to SEQ ID NO: 8 under stringent conditions, and,
(f) a protein consisting of an amino acid sequence encoded by a DNA having the nucleotide sequence shown by SEQ ID NO: 8 wherein 1 or plural nucleotides are substituted, deleted, inserted and/or added, and having a γ-glutamylcysteine synthetase activity.
[7] The production method according to the above-mentioned [5], wherein the glutathione synthetase is selected from the following (a)-(f):
(a) a protein consisting of the amino acid sequence shown by SEQ ID NO: 9,
(b) a protein having the amino acid sequence shown by SEQ ID NO: 9, wherein 1 or plural amino acids are deleted, substituted, inserted and/or added, and having a glutathione synthetase activity,
(c) a protein consisting of an amino acid sequence having 60% or more sequence identity to the amino acid sequence shown by SEQ ID NO: 9,
(d) a protein consisting of an amino acid sequence encoded by a nucleotide sequence shown by SEQ ID NO: 10,
(e) a protein consisting of an amino acid sequence encoded by a DNA that hybridizes with a DNA having a nucleotide sequence complementary to SEQ ID NO: 10 under stringent conditions, and,
(f) a protein consisting of an amino acid sequence encoded by a DNA having the nucleotide sequence shown by SEQ ID NO: 10 wherein 1 or plural nucleotides are substituted, deleted, inserted and/or added, and having a glutathione synthetase activity.
[8] The production method according to any of the above-mentioned [1]-[7], wherein the yeast is a yeast in which the expression of glutathione transport enzyme is enhanced as compared to the parent strain.
[9] The production method according to the above-mentioned [8], wherein the glutathione transport enzyme is selected from the following (a)-(f):
(a) a protein consisting of the amino acid sequence shown by SEQ ID NO: 11,
(b) a protein having the amino acid sequence shown by SEQ ID NO: 11, wherein 1 or plural amino acids are deleted, substituted, inserted and/or added, and having a glutathione transport enzyme activity,
(c) a protein consisting of an amino acid sequence having 60% or more sequence identity to the amino acid sequence shown by SEQ ID NO: 11,
(d) a protein consisting of an amino acid sequence encoded by a nucleotide sequence shown by SEQ ID NO: 12,
(e) a protein consisting of an amino acid sequence encoded by a DNA that hybridizes with a DNA having a nucleotide sequence complementary to SEQ ID NO: 12 under stringent conditions, and,
(f) a protein consisting of an amino acid sequence encoded by a DNA having the nucleotide sequence shown by SEQ ID NO: 12 wherein 1 or plural nucleotides are substituted, deleted, inserted and/or added, and having a glutathione transport enzyme activity.
[10] The production method according to any of the above-mentioned [1]-[9], wherein the yeast is a yeast belonging to the genus Saccharomyces, the genus Candida or the genus Pichia.
[11] A method of producing reduced glutathione, comprising reducing oxidized glutathione in the glutathione obtained by the production method according to any of the above-mentioned [1]-[10].
[12] A yeast having artificially modified genes such that the expression of thiol oxidase is enhanced and the expression of γ-glutamylcysteine synthetase and/or glutathione synthetase is enhanced as compared to the parent strain.
[13] A yeast having artificially modified genes such that the expression of thiol oxidase is enhanced and the expression of glutathione transport enzyme is enhanced as compared to the parent strain.
[14] The yeast according to the above-mentioned [12] or [13], wherein the yeast is a yeast belonging to the genus Saccharomyces, the genus Candida or the genus Pichia.

### Effect of the Invention

According to the method of the present invention, glutathione can be efficiently produced.

### DESCRIPTION OF EMBODIMENTS

Hereinafter, the method of the present invention is explained in detail.

The present invention is a method of producing glutathione by a yeast in which intracellular thiol oxidase activity is increased, and preferably, glutathione reductase activity is reduced.

More preferably, the present invention is a method of producing glutathione by a yeast whose γ-glutamylcysteine synthetase (GSH1) and/or glutathione synthetase (GSH2) and/or glutathione transport enzyme (YCF1) activities are increased, in addition to the aforementioned properties.

In the present invention, “enzyme activity is increased” means that an enzyme activity of interest is increased as compared to that of the parent strain such as wild-type strain and the like. “Enzyme activity is increased” encompasses not only increasing an enzyme activity of interest in a strain natively having the enzyme activity, but also conferring an enzyme activity of interest to a strain natively lacking the enzyme activity.

Increase of enzyme activity can be accomplished by, for example, artificially modifying a gene of a strain. Such modification can be achieved by, for example, enhancing the expression of a gene encoding an enzyme of interest.

Enhancement of gene expression can be achieved by, for example, substituting the promoter of the gene on chromosome with a more potent promoter. The “more potent promoter” means a promoter that improves transcription of a gene as compared to the naturally occurring wild-type promoter. As a more potent promoter, a highly active form of native promoter may be obtained using various reporter genes. Alternatively, as more potent promoters, known high expression promoters, for example, PGK1, PDC1, TDH3, TEF1, HXT7, ADH1 and the like gene may also be used. The substitution with a more potent promoter can be utilized in combination with the below-mentioned increase of copy number of gene. As an example of utilization of a more potent promoter, a method of enhancing γ-glutamylcysteine synthetase activity by substituting the promoter of the γ-glutamylcysteine synthetase gene on chromosome with a promoter having a potent transcriptional activity is disclosed (Yasuyuki Ohtake et al., Bioscience and Industry, 50(10), 989-994, 1992).

Enhancement of gene expression can be achieved by, for example, increasing copy number of the gene.

The copy number of a gene can be increased by introducing the gene of interest onto the chromosome. Introduction of a gene onto the chromosome can be performed, for example, by utilizing homologous recombination. For example, many copies of a gene can be introduced into the chromosome by homologous recombination using a sequence containing many copies in the chromosome as the target. As the sequence containing many copies in the chromosome, autonomously replicating sequence (ARS) consisting of unique short repeat sequences, and rDNA sequence having about 150 copies can be mentioned. Using a plasmid containing ARS, yeast was transformed as described in WO 95/32289. Alternatively, a gene may be incorporated into a transposon, and the transposon may be transferred into the chromosome to introduce many copies of the gene.

In addition, the copy number of a gene can also be increased by introducing a vector containing the gene of interest into a host. As the vector, for example, a plasmid having a replication origin of CEN 4 or a multiple copy type plasmid having a replication origin of 2 μm DNA can be used preferably. The gene of interest may be inserted into a vector in combination with a suitable promoter to achieve expression of the gene of interest. When a vector containing a promoter suitable for expressing the gene is used, the gene of interest may be expressed by utilizing the promoter in the vector.

In addition, a modification to increase enzyme activity can also be achieved by, for example, enhancing the specific activity of the enzyme of interest. An enzyme having an enhanced specific activity can be obtained by, for example, searching through various organisms. Also, a highly active form may be acquired by introducing mutation into native enzymes. The specific activity may be enhanced solely or enhanced in free combination with the above-mentioned method for enhancing gene expression.

Increase in the enzyme activity of interest can be confirmed by measuring the activity of the enzyme. Thiol oxidase activity can be measured by the method described in FEBS Letters 477 (2000) 62-66. γ-Glutamylcysteine synthetase activity can be measured by the method of Jackson (Jackson, R. C., Biochem. J., 111, 309 (1969)). Glutathione synthetase activity can be measured by the method of Gushima et al. (Gushima, T. et al., J. Appl. Biochem., 5, 210 (1983)). Glutathione transport enzyme activity can be measured by reference to THE JOURNAL OF BIOLOGICAL CHEMISTRY Vol. 273, No. 50, Issue of December 11, pp. 33449-33454, 1998.

Increase of the transcription amount of a gene encoding the enzyme of interest can be confirmed by comparing the amount of mRNA transcribed from the gene with that of the parent strain. As a method for evaluating the amount of mRNA, Northern hybridization, RT-PCR and the like can be mentioned (Molecular cloning (Cold spring Harbor Laboratory Press, Cold spring Harbor (USA), 2001)). A preferable increase in the amount of mRNA is, for example, not less than 1.5-fold, not less than 2-fold, or not less than 3-fold, as compared to the parent strain.

Increase of the amount of the enzyme of interest can be confirmed by Western blot using an antibody (Molecular cloning (Cold spring Harbor Laboratory Press, Cold spring Harbor (USA), 2001)). The amount of the enzyme of interest preferably increases to, for example, not less than 1.5-fold, not less than 2-fold, or not less than 3-fold, as compared to the parent strain.

When the yeast of the present invention has polyploidy not less than diploid, and the enzyme activity is increased by modification of chromosome, the yeast of the present invention may heterozygously have a chromosome modified to increase enzymatic activity and a wild-type chromosome, or may be a homozygote of a chromosome modified to increase enzymatic activity, as long as it can accumulate glutathione.

In the present invention, “enzyme activity decreases” means that an enzyme activity of interest decreased as compared to that of the parent strain such as wild-type strain and the like, and includes complete disappearance of the activity.

Decrease of enzyme activity can be accomplished by, for example, artificially modifying a gene of a strain. Such modification can be achieved by, for example, mutagenesis treatment or genetic recombination technique.

As a mutagenesis treatment, UV radiation or treatment with mutagens generally used for mutagenesis treatments such as N-methyl-N′-nitro-N-nitrosoguanidine (MNNG), ethylmethane sulfonate (EMS), methylmethane sulfonate (MMS) and the like can be mentioned.

As a genetic recombination technique, known techniques (FEMS Microbiology Letters 165 (1998) 335-340, JOURNAL OF BACTERIOLOGY, December 1995, p 7171-7177, Curr Genet 1986; 10(8):573-578, WO 98/14600 etc.) can be utilized.

A modification that decreases the enzyme activity can be achieved by, for example, decreasing the expression of a gene encoding the enzyme of interest. Gene expression can be decreased by, for example, modifying an expression control sequence of the gene such as promoter and the like. When an expression control sequence is modified, preferably not less than one nucleotide, more preferably not less than 2 nucleotides, particularly preferably not less than 3 nucleotides in the expression control sequence are modified. In addition, the expression control sequence may be partly or entirely deleted.

A modification to lower the enzyme activity can be achieved by, for example, partly or entirely deleting the coding region of a gene encoding the enzyme of interest on the chromosome. Furthermore, the whole gene including the sequences before and after the gene on the chromosome may be deleted. The region to be deleted may be any region such as N-terminal region, internal region, C-terminal region and the like as long as the enzyme activity can be decreased. Generally, a longer region to be deleted can certainly inactivate the gene. It is preferable that the sequences before and after the region to be deleted do not have the same reading frame.

A modification to lower the enzyme activity can also be achieved by, for example, introducing amino acid substitution (missense mutation), introducing a termination codon (nonsense mutation), or introducing a frame shift mutation that adds or deletes 1 or 2 nucleotides and the like, into the coding region of a gene encoding the enzyme of interest on the chromosome.

In addition, a modification that lowers the enzyme activity can also be achieved by, for example, introducing other sequence into the coding region of a gene encoding the enzyme of interest on the chromosome. While the insertion site may be any region of the gene, a longer region to be inserted can certainly inactivate the gene. It is preferable that the sequences before and after the region to be inserted do not have the same reading frame. While other sequence is not particularly limited as long as it decreases the function of the protein to be encoded or makes the function disappear, for example, a marker gene and a gene useful for the production of γ-glutamyl compounds such as glutathione and the like can be mentioned.

Modification of a gene on the chromosome as mentioned above can be achieved by, for example, producing a deleted gene lacking a partial sequence of the gene and modified to not produce a protein that functions normally, transforming a yeast with a recombinant DNA containing the deleted gene, and substituting the gene on the chromosome by a deleted gene by causing homologous recombination between the deleted gene and the gene on the chromosome. In this case, the operation is facilitated when the recombinant DNA contains a marker gene according to the phenotype of the host such as auxotrophy and the like. In addition, a strain having a recombinant DNA integrated with the chromosome can be efficiently obtained when the aforementioned recombinant DNA is linearized by cleavage with a restriction enzyme and the like. Even when a protein encoded by the deleted gene is produced, it has a steric structure different from that of a wild-type protein and the function thereof decreases or disappears.

Depending on the structure of the recombinant DNA to be used, homologous recombination sometimes results in the insertion of a wild-type gene and a deleted gene into the chromosome, with the other part of the recombinant DNA (e.g., vector part and marker gene) interposed between them. Since the wild-type gene functions in this state, it is necessary to cause homologous recombination again between the two genes, remove one copy of the gene together with the vector part and the marker gene from the chromosome DNA, and select a sequence maintaining the deleted gene.

Alternatively, for example, a yeast is transformed with a linear DNA containing any sequence provided with, on both ends thereof, upstream and downstream sequences of the substitution target site on the chromosome, to cause homologous recombination at each of the upstream and downstream of the substitution target site, whereby the substitution target site can be substituted by said any sequence in one step. As said any sequence, a sequence containing a marker gene can be used. The marker gene may be removed thereafter where necessary. When the marker gene is to be removed, a sequence for homologous recombination may be added to the both ends of the marker gene to enable efficient removal of the marker gene.

Decrease in the enzyme activity of interest can be confirmed by measuring the activity of the enzyme. The activity of glutathione reductase can be measured by a known method (Glutathione Reductase Assay Kit Product No. 7510-100-K manufactured by Cosmo Bio etc.).

Decrease of the transcription amount of a gene encoding the enzyme of interest can be confirmed by comparing the amount of mRNA transcribed from the gene with that of the parent strain. As a method for evaluating the amount of mRNA, Northern hybridization, RT-PCR and the like can be mentioned (Molecular cloning (Cold spring Harbor Laboratory Press, Cold spring Harbor (USA), 2001)). The amount of mRNA preferably decreases to, for example, not more than 50%, not more than 20%, not more than 10%, not more than 5%, or 0%, as compared to the parent strain.

Decrease of the amount of the enzyme of interest can be confirmed by Western blot using an antibody (Molecular cloning (Cold spring Harbor Laboratory Press, Cold spring Harbor (USA), 2001)). The amount of the enzyme of interest preferably decreases to, for example, not more than 50%, not more than 20%, not more than 10%, not more than 5%, or 0%, as compared to the parent strain.

As a transformation method of yeast, a method generally used for yeast transformation such as protoplast method, KU method (H. Ito et al., J. Bateriol., 153-163 (1983)), KUR method (Fermentation and Industry, vol.43, p. 630-637 (1985)), electroporation method (Luis et al., FEMS Micro biology Letters 165 (1998) 335-340), method using carrier DNA (Gietz R. D. and Schiestl R. H., Methods Mol. Cell. Biol. 5:255-269 (1995)) and the like can be adopted. Operations such as yeast spore formation, separation of haploid yeast, and the like are described in “Chemistry and Biology Experimental Line, 31, experiment technique for yeast”, the 1st edition, Hirokawa Shoten; “Bio manual series 10, gene experiment method with yeast” the 1st edition, YODOSHA CO., LTD. and the like.

### (1) Thiol Oxidase

Thiol oxidase is generally an enzyme that catalyzes the reaction of the following formula (1) in vivo.

According to known documents, thiol oxidase is known to be mainly involved in the folding of protein. For example, ERV1, which is one kind of thiol oxidase, activates Mia40 by oxidizing a thiol group contained in Mia40 that introduces a disulfide bond into proteins in the mitochondrial inner membrane (non-patent document, The EMBO Journal (2012) 31, 3169-3182). There has been made no report to date that shows a relationship between thiol oxidase and glutathione production.

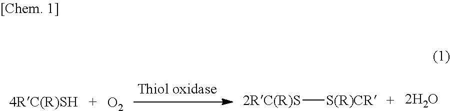

Thiol oxidase in the present invention only needs to have, as mentioned above, an activity to oxidize an intermolecular or intramolecular thiol group of protein or peptide with an oxygen molecule and form a disulfide bond (i.e., thiol oxidase activity), and is not particularly limited. For example, it is preferably any of the following (a)-(f):
(a) a protein consisting of the amino acid sequence shown by SEQ ID NO: 1 or 2,
(b) a protein having the amino acid sequence shown by SEQ ID NO: 1 or 2, wherein 1 or plural amino acids are deleted, substituted, inserted and/or added, and having a thiol oxidase activity,
(c) a protein consisting of an amino acid sequence having 60% or more sequence identity to the amino acid sequence shown by SEQ ID NO: 1 or 2,
(d) a protein consisting of an amino acid sequence encoded by a nucleotide sequence shown by SEQ ID NO: 3 or 4,
(e) a protein consisting of an amino acid sequence encoded by a DNA that hybridizes with a DNA having a nucleotide sequence complementary to SEQ ID NO: 3 or 4 under stringent conditions, and
(f) a protein consisting of an amino acid sequence encoded by a DNA having the nucleotide sequence shown by SEQ ID NO: 3 or 4 wherein 1 or plural nucleotides are substituted, deleted, inserted and/or added, and having a thiol oxidase activity.

A protein having the amino acid sequence shown by SEQ ID NO: 1 or 2, wherein 1 or plural amino acids are substituted, inserted, deleted and/or added can be prepared according to a known method described in “Current Protocols in Molecular Biology (John Wiley and Sons, Inc., 1989)” and the like, and is encompassed in the above-mentioned protein as long as it has a thiol oxidase activity.

An amino acid sequence modified by substitution, insertion, deletion and/or addition may contain only one kind (e.g., substitution) of modification, or two or more kinds of modifications (e.g., substitution and insertion). In the case of substitution, an amino acid used for substitution is preferably an amino acid having properties similar to those of amino acid before substitution (cognate amino acid). As used herein, amino acids in the same group in the following groups are the cognate amino acids.
(group 1: neutral non-polar amino acids) Gly, Ala, Val, Leu, Ile, Met, Cys, Pro, Phe
(group 2: neutral polar amino acids) Ser, Thr, Gln, Asn, Trp, Tyr
(group 3: acidic amino acids) Glu, Asp
(group 4: basic amino acids) His, Lys, Arg

In the above-mentioned description, the plural amino acids mean, for example, not more than 60, preferably 20, more preferably 15, further preferably 10, further preferably 5, 4, 3 or 2 amino acids.

The sequence identity to the amino acid sequence shown in SEQ ID NO: 1-2 is preferably not less than 60%, more preferably not less than 70%, further preferably not less than 80%, further preferably not less than 85%, further preferably not less than 90%, most preferably not less than 95%. The sequence identity to the amino acid sequence is expressed by a value obtained by comparing the amino acid sequence shown in SEQ ID NO: 1-2 and the amino acid sequence desired to be evaluated, dividing the number of positions, at which amino acids matched between the both sequences, by the total number of the compared amino acids, and multiplying the value by 100.

An additional amino acid sequence can be bonded to the amino acid sequence described in SEQ ID NO: 1-2 as long as it has a thiol oxidase activity. In addition, a fusion protein with other protein can also be provided.

The DNA that hybridizes with a DNA having a nucleotide sequence complementary to the nucleotide sequence shown by SEQ ID NO: 3 or 4 under stringent conditions means a DNA obtained by colony hybridization method, plaque hybridization method, or Southern hybridization method and the like under stringent conditions by using a DNA consisting of a nucleotide sequence complementary to the nucleotide sequence shown by SEQ ID NO: 3 or 4 as a probe.

Hybridization can be performed according to the method described in “Molecular Cloning, A laboratory manual, second edition (Cold Spring Harbor Laboratory Press, 1989)” and the like. DNA that hybridizes under stringent conditions is, for example, a DNA obtained by hybridization using a filter immobilizing a colony or plaque-derived DNA in the presence of 0.7-1.0 M NaCl at 65° C., and washing the filter with 2-fold concentration of SSC solution (1×concentration of SSC solution is composed of 150 mM sodium chloride and 15 mM sodium citrate) at 65° C. It is preferably a DNA obtained by washing with 1×concentration of SSC solution at 65° C., more preferably 0.5×concentration of SSC solution at 65° C., further preferably 0.2×concentration of SSC solution at 65° C., most preferably 0.1×concentration of SSC solution at 65° C.

While the hybridization conditions are described above, they are not particularly limited to those conditions. As factors affecting the stringency of hybridization, plural factors such as temperature, salt concentration and the like are considered, and those of ordinary skill in the art can realize the optimal stringency by appropriately determining those factors.

A DNA hybridizable under the above-mentioned conditions is, for example, a DNA having sequence identity of not less than 70%, preferably not less than 74%, more preferably not less than 79%, further preferably not less than 85%, further more preferably not less than 90%, most preferably not less than 95%, to the DNA shown by SEQ ID NO: 3 or 4.

The sequence identity (%) of DNA is expressed by a value obtained by optimally aligning two DNAs to be compared, dividing the number of positions, at which a nucleic acid base (e.g., A, T, C, G, U or I) matched between the both sequences, by the total number of the compared nucleotides, and multiplying the resulting value by 100.

The sequence identity of DNA can be calculated using, for example, the following tools for sequence analysis: GCG Wisconsin Package (Program Manual for The Wisconsin Package, Version 8, September 1994, Genetics Computer Group, 575 Science Drive Medison, Wis., USA 53711; Rice, P. (1996) Program Manual for EGCG Package, Peter Rice, The Sanger Centre, Hinxton Hall, Cambridge, CB10 1RQ, England) and the.e.xPASy World Wide Web Molecule Server for Biology (Geneva University Hospital and University of Geneva, Geneva, Switzerland).

DNA having a nucleotide sequence shown by SEQ ID NO: 3 or 4 wherein 1 or plural nucleotides are substituted, deleted, inserted and/or added can be prepared according to a known method described in “Current Protocols in Molecular Biology (John Wiley and Sons, Inc., 1989)” and the like.

A nucleotide sequence modified by substitution, insertion, deletion and/or addition may contain only one kind (e.g., substitution) of modification, or two or more kinds of modifications (e.g., substitution and insertion).

The plural nucleotides described above are not particularly limited as long as a protein encoded by the DNA has a thiol oxidase activity, and mean, for example, not more than 150, preferably 100, more preferably 50, further to preferably 20, further more preferably 10, 5, 4, 3 or 2 nucleotides.

The representative thiol oxidase in the present invention includes thiol oxidase (ERV1) and thiol oxidase (ERO1).

### (2) Glutathione Reductase

Glutathione reductase is an enzyme having an activity to reduce oxidized glutathione represented by the following formula (2) by utilizing NADPH (reduced nicotinamide dinucleotide phosphate).

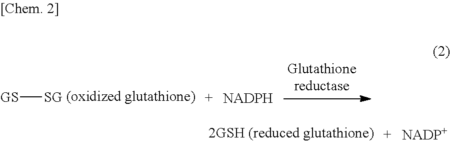

Glutathione reductase in the present invention only needs to have an activity to reduce disulfide bond by utilizing NADPH or NADH (i.e., glutathione reductase activity), and is not particularly limited, and
(a) a protein consisting of the amino acid sequence shown by SEQ ID NO: 5,
(b) a protein having the amino acid sequence shown by SEQ ID NO: 5, wherein 1 or plural amino acids are deleted, substituted, inserted and/or added, and having a glutathione reductase activity,
(c) a protein consisting of an amino acid sequence having 60% or more sequence identity to the amino acid sequence shown by SEQ ID NO: 5,
(d) a protein consisting of an amino acid sequence encoded by a nucleotide sequence shown by SEQ ID NO: 6,
(e) a protein consisting of an amino acid sequence encoded by a DNA that hybridizes with a DNA having a nucleotide sequence complementary to SEQ ID NO: 6 under stringent conditions, and,
(f) a protein consisting of an amino acid sequence encoded by a DNA having the nucleotide sequence shown by SEQ ID NO: 6 wherein 1 or plural nucleotides are substituted, deleted, inserted and/or added, and having a glutathione reductase activity
   can be mentioned.

A protein having the amino acid sequence shown by SEQ ID NO: 5, wherein 1 or plural amino acids are substituted, inserted, deleted and/or added can be prepared according to the method described in the above-mentioned (1), and is encompassed in the above-mentioned protein as long as it has a glutathione reductase activity.

An amino acid sequence modified by substitution, insertion, deletion and/or addition may contain only one kind (e.g., substitution) of modification, or two or more kinds of modifications (e.g., substitution and insertion). In the case of substitution, an amino acid used for substitution is preferably an amino acid having properties similar to those of amino acid before substitution (cognate amino acid). Cognate amino acid is as mentioned above in (1).

In the above-mentioned description, the plural amino acids mean, for example, not more than 60, preferably 20, more preferably 15, further preferably 10, further preferably 5, 4, 3 or 2 amino acids.

The sequence identity to the amino acid sequence shown in SEQ ID NO: 5 is preferably not less than 60%, more preferably not less than 70%, further preferably not less than 80%, further preferably not less than 85%, further preferably not less than 90%, most preferably not less than 95%. The sequence identity to the amino acid sequence can be calculated by the aforementioned method in (1).

An additional amino acid sequence can be bonded to the amino acid sequence described in SEQ ID NO: 5 as long as it has a glutathione reductase activity. In addition, a fusion protein with other protein can also be provided.

The DNA that hybridizes with a DNA having a nucleotide sequence complementary to the nucleotide sequence shown by SEQ ID NO: 6 under stringent conditions means a DNA obtained by colony hybridization method, plaque hybridization method, or Southern hybridization method and the like under stringent conditions by using a DNA consisting of a nucleotide sequence complementary to the nucleotide sequence shown by SEQ ID NO: 6 as a probe. The conditions and the like of hybridization are as mentioned above in (1).

A DNA hybridizable under the above-mentioned conditions is, for example, a DNA having sequence identity of not less than 70%, preferably not less than 74%, more preferably not less than 79%, further preferably not less than 85%, further more preferably not less than 90%, most preferably not less than 95%, to the DNA shown by SEQ ID NO: 6. The sequence identity (%) of the DNA is as mentioned in (1) above.

DNA having a nucleotide sequence shown by SEQ ID NO: 6 wherein 1 or plural nucleotides are substituted, deleted, inserted and/or added can be prepared according to the aforementioned method in (1).

A nucleotide sequence modified by substitution, insertion, deletion and/or addition may contain only one kind (e.g., substitution) of modification, or two or more kinds of modifications (e.g., substitution and insertion).

The plural nucleotides described above are not particularly limited as long as a protein encoded by the DNA has a glutathione reductase activity, and mean, for example, not more than 150, preferably 100, more preferably 50, further preferably 20, further more preferably 10, 5, 4, 3 or 2 nucleotides.

A means for confirming that a protein having the amino acid sequence shown by SEQ ID NO: 5, wherein 1 or plural amino acids are deleted, substituted, inserted and/or added, is a protein having a glutathione reductase activity is, for example, a method including producing a transformant that expresses a protein, whose activity is desired to be confirmed, by a DNA recombinant method, producing the protein by using the transformant, placing the protein, oxidized glutathione and NADPH in an aqueous medium, and analyzing the presence or absence of production and accumulation of reduced glutathione or NADP in the aqueous medium by HPLC and the like.

When the yeast of the present invention has polyploidy not less than diploid, the yeast of the present invention may heterozygously have a gene modified to show a decreased enzyme activity and a wild-type gene as long as it can accumulate γ-glutamyl compounds such as glutathione and the like. However, it is generally preferably a homozygote of a gene modified to show a decreased enzymatic activity.

In the present invention, the glutathione reductase activity is preferably decreased to not more than 50%, more preferably not more than 20%, further preferably not more than 10%, particularly preferably not more than 5%, as compared to the parent strain. Substantial disappearance of the glutathione reductase activity is preferable.

### (3) γ-Glutamylcysteine Synthetase

While γ-glutamylcysteine synthetase in the present invention only needs to have an activity to condense glutamic acid and cysteine to synthesize glutamylcysteine (i.e., γ-glutamylcysteine synthetase activity) and is not particularly limited,
(a) a protein consisting of the amino acid sequence shown by SEQ ID NO: 7,
(b) a protein having the amino acid sequence shown by SEQ ID NO: 7, wherein 1 or plural amino acids are deleted, substituted, inserted and/or added, and having a γ-glutamylcysteine synthetase activity,
(c) a protein consisting of an amino acid sequence having 60% or more sequence identity to the amino acid sequence shown by SEQ ID NO: 7,
(d) a protein consisting of an amino acid sequence encoded by a nucleotide sequence shown by SEQ ID NO: 8,
(e) a protein consisting of an amino acid sequence encoded by a DNA that hybridizes with a DNA having a nucleotide sequence complementary to SEQ ID NO: 8 under stringent conditions, and,
(f) a protein consisting of an amino acid sequence encoded by a DNA having the nucleotide sequence shown by SEQ ID NO: 8 wherein 1 or plural nucleotides are substituted, deleted, inserted and/or added, and having a γ-glutamylcysteine synthetase activity
   can be mentioned.

A protein having the amino acid sequence shown by SEQ ID NO: 7, wherein 1 or plural amino acids are substituted, inserted, deleted and/or added can be prepared according to the method described in the above-mentioned (1), and is encompassed in the above-mentioned protein as long as it has a γ-glutamylcysteine synthetase activity.

An amino acid sequence modified by substitution, insertion, deletion and/or addition may contain only one kind (e.g., substitution) of modification, or two or more kinds of modifications (e.g., substitution and insertion). In the case of substitution, an amino acid used for substitution is preferably an amino acid having properties similar to those of amino acid before substitution (cognate amino acid). Cognate amino acid is as mentioned above in (1).

In the above-mentioned description, the plural amino acids mean, for example, not more than 60, preferably 20, more preferably 15, further preferably 10, further preferably 5, 4, 3 or 2 amino acids.

The sequence identity to the amino acid sequence shown in SEQ ID NO: 7 is preferably not less than 60%, more preferably not less than 70%, further preferably not less than 80%, further preferably not less than 85%, further preferably not less than 90%, most preferably not less than 95%. The sequence identity to the amino acid sequence can be calculated by the aforementioned method in (1).

An additional amino acid sequence can be bonded to the amino acid sequence described in SEQ ID NO: 7 as long as it has a γ-glutamylcysteine synthase activity. In addition, a fusion protein with other protein can also be provided.

The DNA that hybridizes with a DNA having a nucleotide sequence complementary to the nucleotide sequence shown by SEQ ID NO: 8 under stringent conditions means a DNA obtained by colony hybridization method, plaque hybridization method, or Southern hybridization method and the like under stringent conditions by using a DNA consisting of a nucleotide sequence complementary to the nucleotide sequence shown by SEQ ID NO: 8 as a probe. The conditions and the like of hybridization are as mentioned above in (1).

A DNA hybridizable under the above-mentioned conditions is, for example, a DNA having sequence identity of not less than 70%, preferably not less than 74%, more preferably not less than 79%, further preferably not less than 85%, further more preferably not less than 90%, most preferably not less than 95%, to the DNA shown by SEQ ID NO: 8. The sequence identity (%) of the DNA is as mentioned in (1) above.

DNA having a nucleotide sequence shown by SEQ ID NO: 8 wherein 1 or plural nucleotides are substituted, deleted, inserted and/or added can be prepared according to the aforementioned method in (1).

A nucleotide sequence modified by substitution, insertion, deletion and/or addition may contain only one kind (e.g., substitution) of modification, or two or more kinds of modifications (e.g., substitution and insertion).

The plural nucleotides described above are not particularly limited as long as a protein encoded by the DNA has a γ-glutamylcysteine synthetase activity, and mean, for example, not more than 150, preferably 100, more preferably 50, further preferably 20, further more preferably 10, 5, 4, 3 or 2 nucleotides.

A means for confirming that a protein having the amino acid sequence shown by SEQ ID NO: 7, wherein 1 or plural amino acids are deleted, substituted, inserted and/or added, is a protein having a γ-glutamylcysteine synthetase activity is, for example, a method including producing a transformant that expresses a protein, whose activity is desired to be confirmed, by a DNA recombinant method, producing the protein by using the transformant, placing the protein, L-glutamic acid and L-cysteine in an aqueous medium, and analyzing the presence or absence of production and accumulation of γ-glutamylcysteine in the aqueous medium by HPLC and the like.

As the γ-glutamylcysteine synthetase in the present invention, GSH1 shown by SEQ ID NO: 7 is preferable from among the above-mentioned proteins.

### (4) Glutathione Synthetase

While glutathione synthetase in the present invention only needs to have an activity to condense γ-glutamylcysteine and glycine to synthesize glutathione (i.e., glutathione synthetase activity) and is not particularly limited,
(a) a protein consisting of the amino acid sequence shown by SEQ ID NO: 9,
(b) a protein having the amino acid sequence shown by SEQ ID NO: 9, wherein 1 or plural amino acids are deleted, substituted, inserted and/or added, and having a glutathione synthetase activity,
(c) a protein consisting of an amino acid sequence having 60% or more sequence identity to the amino acid sequence shown by SEQ ID NO: 9,
(d) a protein consisting of an amino acid sequence encoded by a nucleotide sequence shown by SEQ ID NO: 10,
(e) a protein consisting of an amino acid sequence encoded by a DNA that hybridizes with a DNA having a nucleotide sequence complementary to SEQ ID NO: 10 under stringent conditions, and,
(f) a protein consisting of an amino acid sequence encoded by a DNA having the nucleotide sequence shown by SEQ ID NO: 10 wherein 1 or plural nucleotides are substituted, deleted, inserted and/or added, and having a glutathione synthetase activity
   can be mentioned.

A protein having the amino acid sequence shown by SEQ ID NO: 9, wherein 1 or plural amino acids are substituted, inserted, deleted and/or added can be prepared according to the method described in the above-mentioned (1), and is encompassed in the above-mentioned protein as long as it has a glutathione synthetase activity.

An amino acid sequence modified by substitution, insertion, deletion and/or addition may contain only one kind (e.g., substitution) of modification, or two or more kinds of modifications (e.g., substitution and insertion). In the case of substitution, an amino acid used for substitution is preferably an amino acid having properties similar to those of amino acid before substitution (cognate amino acid). Cognate amino acid is as mentioned above in (1).

In the above-mentioned description, the plural amino acids mean, for example, not more than 60, preferably 20, more preferably 15, further preferably 10, further preferably 5, 4, 3 or 2 amino acids.

The sequence identity to the amino acid sequence shown in SEQ ID NO: 9 is preferably not less than 60%, more preferably not less than 70%, further preferably not less than 80%, further preferably not less than 85%, further preferably not less than 90%, most preferably not less than 95%. The sequence identity to the amino acid sequence can be calculated by the aforementioned method in (1).

An additional amino acid sequence can be bonded to the amino acid sequence described in SEQ ID NO: 9 as long as it has a glutathione synthetase activity. In addition, a fusion protein with other protein can also be provided.

The DNA that hybridizes with a DNA having a nucleotide sequence complementary to the nucleotide sequence shown by SEQ ID NO: 10 under stringent conditions means a DNA obtained by colony hybridization method, plaque hybridization method, or Southern hybridization method and the like under stringent conditions by using a DNA consisting of a nucleotide sequence complementary to the nucleotide sequence shown by SEQ ID NO: 10 as a probe. The conditions and the like of hybridization are as mentioned above in (1).

A DNA hybridizable under the above-mentioned conditions is, for example, a DNA having sequence identity of not less than 70%, preferably not less than 74%, more preferably not less than 79%, further preferably not less than 85%, further more preferably not less than 90%, most preferably not less than 95%, to the DNA shown by SEQ ID NO: 10. The sequence identity (%) of the DNA is as mentioned in (1) above.

DNA having a nucleotide sequence shown by SEQ ID NO: 10 wherein 1 or plural nucleotides are substituted, deleted, inserted and/or added can be prepared according to the aforementioned method in (1).

A nucleotide sequence modified by substitution, insertion, deletion and/or addition may contain only one kind (e.g., substitution) of modification, or two or more kinds of modifications (e.g., substitution and insertion).

The plural nucleotides described above are not particularly limited as long as a protein encoded by the DNA has a glutathione synthetase activity, and mean, for example, not more than 150, preferably 100, more preferably 50, further preferably 20, further more preferably 10, 5, 4, 3 or 2 nucleotides.

A means for confirming that a protein having the amino acid sequence shown by SEQ ID NO: 9, wherein 1 or plural amino acids are deleted, substituted, inserted and/or added, is a protein having a glutathione synthetase activity is, for example, a method including producing a transformant that expresses a protein, whose activity is desired to be confirmed, by a DNA recombinant method, producing the protein by using the transformant, placing the protein, γ-glutamylcysteine and glycine in an aqueous medium, and analyzing the presence or absence of production and accumulation of glutathione in the aqueous medium by HPLC and the like.

As the glutathione synthetase in the present invention, GSH2 shown by SEQ ID NO: 9 is preferable.

### (5) Glutathione Transport Enzyme

In the present invention, the glutathione transport enzyme means a protein having a function to transport glutathione in the cytoplasm to vacuole (i.e., glutathione transport enzyme activity), and is not particularly limited as long as it has such function.

More preferably, as the glutathione transport enzyme,
(a) a protein consisting of the amino acid sequence shown by SEQ ID NO: 11,
(b) a protein having the amino acid sequence shown by SEQ ID NO: 11, wherein 1 or plural amino acids are deleted, substituted, inserted and/or added, and having a glutathione transport enzyme activity,
(c) a protein consisting of an amino acid sequence having 60% or more sequence identity to the amino acid sequence shown by SEQ ID NO: 11,
(d) a protein consisting of an amino acid sequence encoded by a nucleotide sequence shown by SEQ ID NO: 12,
(e) a protein consisting of an amino acid sequence encoded by a DNA that hybridizes with a DNA having a nucleotide sequence complementary to SEQ ID NO: 12 under stringent conditions, and,
(f) a protein consisting of an amino acid sequence encoded by a DNA having the nucleotide sequence shown by SEQ ID NO: 12 wherein 1 or plural nucleotides are substituted, deleted, inserted and/or added, and having a glutathione transport enzyme activity
   can be mentioned.

A protein having the amino acid sequence shown by SEQ ID NO: 11, wherein 1 or plural amino acids are substituted, inserted, deleted and/or added can be prepared according to the method described in the above-mentioned (1), and is encompassed in the above-mentioned protein as long as it has a glutathione synthetase activity.

An amino acid sequence modified by substitution, insertion, deletion and/or addition may contain only one kind (e.g., substitution) of modification, or two or more kinds of modifications (e.g., substitution and insertion). In the case of substitution, an amino acid used for substitution is preferably an amino acid having properties similar to those of amino acid before substitution (cognate amino acid). Cognate amino acid is as mentioned above in (1).

In the above-mentioned description, the plural amino acids mean, for example, not more than 60, preferably 20, more preferably 15, further preferably 10, further preferably 5, 4, 3 or 2 amino acids.

The sequence identity to the amino acid sequence shown in SEQ ID NO: 11 is preferably not less than 60%, more preferably not less than 70%, further preferably not less than 80%, further preferably not less than 85%, further preferably not less than 90%, most preferably not less than 95%. The sequence identity to the amino acid sequence can be calculated by the aforementioned method in (1).

An additional amino acid sequence can be bonded to the amino acid sequence described in SEQ ID NO: 11 as long as it has a glutathione transport enzyme activity. In addition, a fusion protein with other protein can also be provided.

The DNA that hybridizes with a DNA having a nucleotide sequence complementary to the nucleotide sequence shown by SEQ ID NO: 12 under stringent conditions means a DNA obtained by colony hybridization method, plaque hybridization method, or Southern hybridization method and the like under stringent conditions by using a DNA consisting of a nucleotide sequence complementary to the nucleotide sequence shown by SEQ ID NO: 12 as a probe. The conditions and the like of hybridization are as mentioned above in (1).

A DNA hybridizable under the above-mentioned conditions is, for example, a DNA having sequence identity of not less than 70%, preferably not less than 74%, more preferably not less than 79%, further preferably not less than 85%, further more preferably not less than 90%, most preferably not less than 95%, to the DNA shown by SEQ ID NO: 12. The sequence identity (%) of the DNA is as mentioned in (1) above.

DNA having a nucleotide sequence shown by SEQ ID NO: 12 wherein 1 or plural nucleotides are substituted, deleted, inserted and/or added can be prepared according to the aforementioned method in (1).

A nucleotide sequence modified by substitution, insertion, deletion and/or addition may contain only one kind (e.g., substitution) of modification, or two or more kinds of modifications (e.g., substitution and insertion).

The plural nucleotides described above are not particularly limited as long as a protein encoded by the DNA has a glutathione transport enzyme activity, and mean, for example, not more than 150, preferably 100, more preferably 50, further preferably 20, further more preferably 10, 5, 4, 3 or 2 nucleotides.

As the glutathione transport enzyme in the present invention, YCF1 shown by SEQ ID NO: 11 is preferable.

### (6) Yeast of the Present Invention

The yeast of the present invention is not particularly limited as long as it has glutathione producibility. For example, yeasts belonging to the genus Saccharomyces such as Saccharomyces cerevisiae, Saccharomyces carlesbergensis, Saccharomyces fragilis, Saccharomyces rouxii and the like, the genus Candida such as Candida utilis, Candida tropicalis and the like, the genus Schizosaccaromyces such as Schizosaccaromyces pombe and the like, the genus Toluropsis such as Toluropsis versatilis, Toluropsis petrophilum and the like, the genus Pichia, the genus Brettanomyces, the genus Mycotorula, the genus Rhodotorula, the genus Hansenula, the genus Endomyces and the like can be mentioned.

Of these, a yeast belonging to the genus Saccharomyces, the genus Candida, or the genus Pichia is preferable, and further, Saccharomyces cerevisiae belonging to the genus Saccharomyces or Candida utilis belonging to the genus Candida is preferable.

### (7) Production Method of Glutathione in the Present Invention

The yeast of the present invention can be cultured in the same manner as in the general culture of microorganisms. That is, any of synthesis medium, semisynthesis medium and natural (composite) medium can be used as long as it appropriately contains carbon source, nitrogen source, inorganic substance, other nutritions and the like.

As the carbon source, various carbohydrate materials such as glucose, glycerol, fructose, sucrose, maltose, mannose, mannitol, xylose, galactose, starch, starch hydrolysate solution, molasses and the like can be used. In addition, various organic acids such as pyruvic acid, acetic acid, lactic acid and the like, and various amino acids such as aspartic acid, alanine and the like can also be used.

As the nitrogen source, various inorganic and organic ammonium salts such as ammonia or ammonium chloride, ammonium phosphate, ammonium sulfate, ammonium nitrate, ammonium carbonate, ammonium acetate and the like, urea and other nitrogen-containing compounds, as well as nitrogenous organic substances such as peptone, NZ amine, meat extract, yeast extract, corn steep liquor, casein hydrolysate, fish meal or digest thereof, defatted soybean or digest and hydrolysate thereof, and the like, and various amino acids such as aspartic acid, glutamic acid, threonine and the like can be used.

As the inorganic substance, moreover, potassium monohydrogen phosphate, potassium dihydrogen phosphate, magnesium sulfate, sodium chloride, ferrous sulfate, manganese sulfate, calcium carbonate and the like can be used.

Culturing is performed under aerobic culture conditions such as shaking culture, aeration-agitation submerged culture and the like. During culturing, pH is preferably adjusted to 3.0-8.0, more preferably 4.0-6.0, most preferably 5.0. As a neutralizing agent for pH control, aqueous ammonia, sodium hydroxide, ammonium carbonate and the like can be used. The amount of air supply to the medium in aerobic culture is preferably not less than 0.2 L/min, more preferably not less than 0.5 L/min, further preferably not less than 1 L/min, per 1 L of the medium. When a jar fermentor with a total volume of 2 L is used for culturing, aerobic culture conditions include the above-mentioned quantity of airflow, and the number of stirring of preferably not less than 200 rpm, more preferably not less than 300 rpm, further preferably not less than 400 rpm. The temperature during culturing is 20-45° C., preferably 25-35° C., most preferably 28-32° C. The culture period is generally 16 hr-72 hr, preferably 24 hr-48 hr. The initial concentration of the cell in the medium (inoculated concentration) varies depending on the kind of the yeast, medium composition and the like, and the initial turbidity (OD600) is preferably 0.01-2.0, more preferably 0.02-1.0, further preferably 0.1-0.4. As for the above-mentioned carbon source such as glucose and the like, both a batch type including charging them all at once in an initial stage of culture and performing culturing, and a fed-batch type including adding them by small portions throughout the culture period are applicable. According to the study results by the present inventors, the fed-batch type is more preferable since it improves the growth rate, makes the final cell concentration higher, and increases the activities of intracellular glutathione synthesis-related enzymes, though subject to change depending on the microorganism to be used. As an index for determining the feeding rate of the carbon source such as glucose and the like in the fed-batch type, turbidity of the cultured broth, oxygen consumption rate, carbon dioxide generation rate, consumption amount of neutralizing agent for pH adjustment and the like can be utilized effectively. By selecting an appropriate index for the yeast to be used, and changing the feeding rate of the carbon source in response to the change, the yeast culture can be optimized and the final cell concentration and the activity of glutathione synthesis-related enzymes can be improved, as a result of which the producibility of glutathione can be actually improved.

The present method affords a cultured broth composed of a yeast mainly containing a high concentration of glutathione in the cells and medium components. The yeast cells in the cultured broth are separated from the medium components by m filtration and centrifugation operations, and glutathione can be extracted from the obtained yeast cells by hot water extraction, alkali extraction method, enzymatic degradation method, autodigestion method, mechanical disruption operation and the like as necessary in an appropriately combination. In addition, the above-mentioned extraction operation may be directly applied to the cultured broth after culturing, or enzymatic degradation method, autodigestion method, or mechanical disruption operation may be performed to elute glutathione in the cells into the medium, and glutathione may be extracted by the above-mentioned extraction operation. A fraction highly containing glutathione or a glutathione powder can be obtained from the thus-obtained glutathione extract by purifying glutathione according to a general method.

It is also possible to obtain glutathione containing a reduced type at a higher proportion by once reducing oxidized glutathione contained in the above-mentioned glutathione extract or the glutathione powder. While the reduction method is not particularly limited, reduction by glutathione reductase is preferably mentioned.

The present invention is explained in more detail in the following by referring to Examples and the like, which are not to be construed as limitative.

### EXAMPLES

### Production Example 1

### Yeast whose Expression of GSH1 Gene and GSH2 Gene is Enhanced (GSH1-Enhanced+GSH2-Enhanced Strain)

Saccharomyces cerevisiae YPH499/GSH1, GSH2 strain in which GSH1 and GSH2 are enhanced, which is described in a non-patent document (Hara K Y, Kiriyama K, Inagaki A, Nakayama H, Kondo A. (2012) Improvement of glutathione production by metabolic engineering the sulfate assimilation pathway of Saccharomyces cerevisiae. Appl Microbiol Biotechnol, 426(2):129-33.), was used.

### Production Example 2

### Preparation of Yeast in which Expression of GSH1 Gene and GSH2 Gene is Enhanced and Expression of ERV1 Gene or ERO1 Gene is Enhanced (GSH1-Enhanced+GSH2-Enhanced+ERV1-Enhanced Strain, GSH1-Enhanced+GSH2-Enhanced+ERO1-Enhanced Strain)

Using genomic DNA of Saccharomyces cerevisiae YPH499 strain as a template and 5′-GGCCGCTAGCATGAAAGCAATAGATAAAATGACGG-3′ (SEQ ID NO: 13) and 5′-GGCCGGATCCTTATTCGTCCCAGCCGTCCTTCC-3′ (SEQ ID NO: 14) as primers, PCR amplification was performed, and the amplification product was digested with NheI and BamEI to give a thiol oxidase (ERV1) gene NheI-BamHI fragment. The fragment was ligated to the NheI-BamHI digestion site of pGK406 described in a non-patent document (J. Biochem. (2009)145: 701-708) to give ERV1-expression plasmid pGK406-ERV1. The obtained plasmid pGK406-ERV1 was digested with restriction enzyme NcoI. Saccharomyces cerevisiae YPH499/GSH1, GSH2 strain shown in Production Example 1 as host yeast strain was transformed to give transformant YPH499/GSH1, GSH2, ERV1 strain (GSH1-enhanced+GSH2-enhanced+ERV1-enhanced strain).

Using genomic DNA of Saccharomyces cerevisiae YPH499 strain as a template and 5′-GGCCGCTAGCATGAGATTAAGAACCGCCATTGCCAC-3′ (SEQ ID NO: 15) and 5′-GGCCGGATCCTTATTGTATATCTAGCTTATAGGAAATAGGC-3′ (SEQ ID NO: 16) as primers, PCR amplification was performed, and the amplification product was digested with NheI and BamHI to give a thiol oxidase (ERO1) gene NheI-BamHI fragment. The fragment was ligated to the NheI-BamHI digestion site of pGK406 described in a non-patent document (J. Biochem. (2009)145: 701-708) to give ERO1-expression plasmid pGK406-ERO1. The obtained plasmid pGK406-ERO1 was digested with restriction enzyme NcoI. Saccharomyces cerevisiae YPH499/GSH1, GSH2 strain shown in Production Example 1 as host yeast strain was transformed to give transformant YPH499/GSH1, GSH2, ERO1 strain (GSH1-enhanced+GSH2-enhanced+ERO1-enhanced strain).

### Production Example 3

### Preparation of Strain in which Expression of GSH1 Gene and GSH2 Gene is Enhanced, and GLR1 Gene is Destructed (GSH1-Enhanced+GSH2-Enhanced+GLR1-Destructed Strain)

Saccharomyces cerevisiae YPH499/GLR1, GSH1, GSH2 strain, in which expression of GSH1 gene and GSH2 gene is enhanced, and GLR1 gene was destructed (GSH1-enhanced+GSH2-enhanced+GLR1-destructed strain), was obtained by the method described in a non-patent document (Kiriyama K, Hara K Y, Kondo A. (2013) Oxidized glutathione fermentation using Saccharomyces cerevisiae engineered for glutathione metabolism. Appl Microbiol Biotechnol, 97(16):7399-7404.).

### Production Example 4

### Preparation of Strain in which Expression of GSH1 Gene and GSH2 Gene is Enhanced, Expression of GLR1 Gene is Decreased, and Expression of ERV1 gene or ERO1 Gene is Enhanced (GSH1-Enhanced+GSH2-Enhanced+GLR1-Destructed+ERV1-Enhanced Strain, GSH1-Enhanced+GSH2-Enhanced+GLR1-Destructed+ERO1-Enhanced Strain)

ERV1-expression plasmid pGK406-ERV1 and ERO1-expression plasmid pGK406-ERO1 shown in Production Example 2 were digested with restriction enzyme NcoI. Saccharomyces cerevisiae YPH499/ΔGLR1, GSH1, GSH2 strain shown in Production Example 3 as host yeast strain was transformed to give transformant YPH499/ΔGLR1, GSH1, GSH2, ERV1 strain (GSH1-enhanced+GSH2-enhanced+GLR1-destructed+ERV1-enhanced strain) and YPH499/ΔGLR1, GSH1, GSH2, ERO1 strain (GSH1-enhanced+GSH2-enhanced+GLR1-destructed+ERO1-enhanced strain), respectively.

### Production Example 5

### Preparation of Strain in which Expression of ERV1 is Enhanced, Expression of GSH1, GSH2 and YCF1 is Further Enhanced and GLR1 Gene is Destructed (GSH1-Enhanced+GSH2-Enhanced+GLR1-Destructed+ERV1-Enhanced+YCF1-Enhanced Strain), and Strain in which Expression of GSH1, GSH2 and YCF1 is Enhanced, and GLR1 Gene is Destructed (GSH1-Enhanced+GSH2-Enhanced+GLR1-Destructed+YCF1-Enhanced Strain)

Using genomic DNA of Saccharomyces cerevisiae YPH499 strain as a template and 5′-AAAAGGATCCATGGCTGGTAATCTTGTTTCATGGGCC-3′ (SEQ ID NO: 17) and 5′-AAAACTCGAGTTAATTTTCATTGACCAAACCAGCCTCC-3′ (SEQ ID NO: 18) as primers, PCR amplification was performed, and the amplification product was digested with BamHI and XhoI to give a glutathione transport enzyme (YCF1) gene BamHI-XhoI fragment. The fragment was ligated to the BamHI-XhoI digestion site of p427TEF (manufactured by Cosmo Bio) to give YCF1 expression plasmid p427-YCF1. Saccharomyces cerevisiae YPH499/GLR1, GSH1, GSH2, ERV1 strain shown in Production Example 4, and Saccharomyces cerevisiae YPH499/ΔGLR1, GSH1, GSH2 strain shown in Production Example 3 as host yeast strains were transformed with p427TEF-YCF1 to give transformant YPH499/ΔGLR1, GSH1, GSH2, ERV1, YCF1 strain (GSH1-enhanced+GSH2-enhanced+GLR1-destructed+ERV1-enhanced+YCF1-enhanced strain) and YPH499/ΔGLR1, GSH1, GSH2, YCF1 strain (GSH1-enhanced+GSH2-enhanced+GLR1-destructed+YCF1-enhanced strain), respectively.

### Example 1

### Production of Glutathione by Yeast in which Expression of GSH1 Gene and GSH2 Gene is Enhanced, and Expression of ERV1 Gene or ERO1 Gene is further Enhanced (GSH1-Enhanced+GSH2-Enhanced+ERV1-Enhanced Strain, GSH1-Enhanced GSH2-Enhanced+ERO1-Enhanced Strain)

Recombinant yeasts YPH499/GSH1, GSH2, ERV1 and YPH499/GSH1, GSH2, ERO1, in which expression of GSH1 and GSH2 is enhanced and expression of ERV1 or ERO1 is enhanced, which were obtained in Production Example 2, were subjected to seed culture by shaking in SD medium (6.7 g/L yeast nitrogen base w/o amino acids (manufactured by Difco laboratories), 20 g/L glucose) (5 ml) at 30° C. for 16-24 hr.

Then, they were inoculated to erlenmeyer flask with baffles containing YPD medium (10 g/L yeast extract (manufactured by Difco laboratories), 20 g/L polypeptone (manufactured by Wako Pure Chemical Industries, Ltd.), 20 g/L glucose (manufactured by Nacalai Tesque)) (20 ml) at OD600=0.03, cultured under conditions of 30° C., agitation 150 rpm for 24 hr, and 1 ml of the cultured broth was recovered.

The cells collected by centrifugation were rinsed twice with sterilized water, and heat treated at 95° C. for 3 min to elute intracellular glutathione. The glutathione concentration of the supernatant centrifuged at 25° C., 20000×g for 5 min was analyzed by the HPLC method and the glutathione concentration per medium was determined.

The cell concentration was determined by measuring the absorbance at 600 nm, or calculated by standing the cultured broth at 80° C. for not less than 12 hr, measuring the weight, determining the dry cell weight by subtracting the weight of the container weighed in advance, and dividing the resulting weight by the amount of the cultured broth.

The glutathione content was calculated by dividing the above-mentioned glutathione concentration by the cell concentration to give glutathione weight per dry cell (glutathione content).

As is clear from Table 1, the glutathione (reduced glutathione+oxidized glutathione) content was improved by enhancing the expression of ERV1 or ERO1 gene.

### Comparative Example 1

### Production of Glutathione by Yeast whose Expression of GSH1 Gene and GSH2 Gene is Enhanced

By a method similar to that in Example 1 except that recombinant yeast Saccharomyces cerevisiae YPH499/GSH1, GSH2 strain, whose expression of GSH1 and GSH2 is enhanced, which was obtained in Production Example 1, was used as a yeast, culturing was performed, and the weight of glutathione per dry cell was calculated. The results are shown in Table 1.

TABLE 1

| | | GSH content (wt %) | | |
|---|---|---|---|---|
| | | | | Total |
| | | Reduced | Oxidized | (reduced + |
| | strain | form | form | oxidized) |
| Comparative | GSH1-enhanced + | 1.91 | 0.73 | 2.64 |
| Example 1 | GSH2-enhanced | | | |
| | strain | | | |
| Example 1 | GSH1-enhanced + | 1.96 | 0.85 | 2.81 |
| | GSH2-enhanced + | | | |
| | ERV1-enhanced | | | |
| | strain | | | |
| | GSH1-enhanced + | 1.78 | 1.04 | 2.82 |
| | GSH2-enhanced + | | | |
| | ERO1-enhanced | | | |
| | strain | | | |

### Example 2

### Production of Glutathione by Strain in which Expression of GSH1 Gene and GSH2 Gene is Enhanced, Expression of GLR1 Gene is Decreased, and Expression of ERV1 gene or ERO1 Gene is Further Enhanced (GSH1-Enhanced+GSH2-enhanced+GLR1-Destructed+ERV1-Enhanced Strain, GSH1-Enhanced+GSH2-Enhanced+GLR1-Destructed+ERO1-Enhanced Strain)

The recombinant yeast YPH499/ΔGLR1, GSH1, GSH2, ERV1 strain and YPH499/ΔGLR1, GSH1, GSH2, ERO1 strain in which expression of GSH1 gene and GSH2 gene is enhanced, expression of GLR1 gene is decreased, and expression of ERV1 gene or ERO1 gene is further enhanced, which was obtained in Production Example 4, were subjected to seed culture by shaking in 5 ml of SD medium (6.7 g/L yeast nitrogen base w/o amino acids (manufactured by Difco laboratories) 20 g/L glucose) containing 0.5 μg/L aureobasidin A at 30° C. for 16-24 hr.

Then, they were inoculated to erlenmeyer flask with baffles containing YPD medium (10 g/L yeast extract (manufactured by Difco laboratories), 20 g/L polypeptone (manufactured by Wako Pure Chemical Industries, Ltd.), 20 g/L glucose (manufactured by Nacalai Tesque)) (20 ml) at OD600=0.03, cultured under conditions of 30° C., agitation 150 rpm for 24 hr, and intracellular glutathione content was measured by the method described in Example 1.

### Comparative Example 2

### Production of Glutathione by Yeast in which Expression of GSH1 Gene and GSH2 Gene is Enhanced, and GLR1 Gene is Destructed

By a method similar to that in Example 2 except that Saccharomyces cerevisiae YPH499/ΔGLR1, GSH1, GSH2 strain, in which expression of GSH1 gene and GSH2 gene is enhanced, and GLR1 gene is destructed, which was obtained in Production Example 3, was used as a yeast, culturing was performed, and the weight of glutathione per dry cell was calculated. The results are shown in Table 2.

TABLE 2

| | | GSH content (wt %) | | |
|---|---|---|---|---|
| | | | | Total |
| | | Reduced | Oxidized | (reduced + |
| | strain | form | form | oxidized) |
| Example 1 | GSH1-enhanced + | 1.96 | 0.85 | 2.81 |
| | GSH2-enhanced + | | | |
| | ERV1-enhanced | | | |
| | strain | | | |
| | GSH1-enhanced + | 1.78 | 1.04 | 2.82 |
| | GSH2-enhanced + | | | |
| | ERO1-enhanced | | | |
| | strain | | | |
| Comparative | GSH1-enhanced + | 1.43 | 1.23 | 2.66 |
| Example 2 | GSH2-enhanced + | | | |
| | GLR1-destructed | | | |
| | strain | | | |
| Example 2 | GSH1-enhanced + | 1.19 | 2.44 | 3.63 |
| | GSH2-enhanced + | | | |
| | GLR1-destructed + | | | |
| | ERV1-enhanced | | | |
| | strain | | | |
| | GSH1-enhanced + | 1.22 | 1.81 | 3.03 |
| | GSH2-enhanced + | | | |
| | GLR1-destructed + | | | |
| | ERO1-enhanced | | | |
| | strain | | | |

As is clear from Table 2, the glutathione content per cell was further improved by not only enhancing the expression of ERV1 gene or ERO1 gene but also destructing GLR1 gene.

### Example 3

### Production of Glutathione by Strain in which ERV1 is Enhanced, Expression of GSH1, GSH2 and YCF1 is Further Enhanced, and GLR1 Gene is Destructed (GGSH1-Enhanced+GSH2-Enhanced+ERV1-Enhanced+YCF1-Enhanced+GLR1-Destructed Strain)

The recombinant yeast YPH499/ΔGLR1, GSH1, GSH2, ERV1, YCF1 strain in which expression of ERV1 is enhanced, expression of GSH1, GSH2 and YCF1 is enhanced, and GLR1 gene is destructed, which was obtained in Production Example 5, was subjected to seed culture by shaking in 5 ml of SD medium (6.7 g/L yeast nitrogen base w/o amino acids (manufactured by Difco laboratories), 20 g/L glucose) containing 0.5 μg/L aureobasidin A at 30° C. for 16-24 hr.

Then, they were inoculated to erlenmeyer flask with baffles containing YPD medium (10 g/L yeast extract (manufactured by Difco laboratories), 20 g/L polypeptone (manufactured by Wako Pure Chemical Industries, Ltd.), 20 g/L glucose (manufactured by Nacalai Tesque)) (20 ml) at OD600=0.03, cultured under conditions of 30° C., agitation 150 rpm for 24 hr, and intracellular glutathione concentration was measured by the method described in Example 1.

### Comparative Example 3

### Production of Glutathione by Yeast in which Expression of GSH1, GSH2 and YCF1 is Enhanced, and GLR1 gene is destructed

By a method similar to that in Example 3 except that Saccharomyces cerevisiae YPH499/ΔGLR1, GSH1, GSH2, YCF1 strain, in which expression of GSH1, GSH2 and YCF1 is enhanced, and GLR1 gene is destructed, which was obtained in Production Example 5, was used as a yeast, culturing was performed, and the weight of glutathione per dry cell was calculated. The results are shown in Table 3.

TABLE 3

| | | GSH content (wt %) | | |
|---|---|---|---|---|
| | | | | Total |
| | | Reduced | Oxidized | (reduced + |
| | strain | form | form | oxidized) |
| Example 2 | GSH1-enhanced + | 1.19 | 2.44 | 3.63 |
| | GSH2-enhanced + | | | |
| | GLR1-destructed + | | | |
| | ERV1-enhanced | | | |
| | strain | | | |
| Comparative | GSH1-enhanced + | 1.90 | 1.65 | 3.55 |
| Example 3 | GSH2-enhanced + | | | |
| | GLR1-destructed + | | | |
| | YCF1-enhanced | | | |
| | strain | | | |
| Example 3 | GSH1-enhanced + | 0.63 | 4.40 | 5.03 |
| | GSH2-enhanced + | | | |
| | GLR1-destructed + | | | |
| | ERV1-enhanced + | | | |
| | YCF1-enhanced | | | |
| | strain | | | |

As is clear from Table 3, the glutathione content was further improved by enhancing the expression of both ERV1 gene and YCF1 gene.

### SEQUENCE LISTING FREE TEXT

SEQ ID NO: 1: amino acid sequence of thiol oxidase (ERV1)
SEQ ID NO: 2: amino acid sequence of thiol oxidase (ERO1)
SEQ ID NO: 3: nucleotide sequence of nucleic acid encoding thiol oxidase (ERV1)
SEQ ID NO: 4: nucleotide sequence of nucleic acid encoding thiol oxidase (ERO1)
SEQ ID NO: 5: amino acid sequence of glutathione reductase (GLR1)
SEQ ID NO: 6: nucleotide sequence of nucleic acid encoding glutathione reductase (GLR1)
SEQ ID NO: 7: amino acid sequence of γ-glutamylcysteine synthase (GSH1)
SEQ ID NO: 8: nucleotide sequence of nucleic acid encoding γ-glutamylcysteine synthase (GSH1)
SEQ ID NO: 9: amino acid sequence of glutathione synthase (GSH2)
SEQ ID NO: 10: nucleotide sequence of nucleic acid encoding glutathione synthase (GSH2)
SEQ ID NO: 11: amino acid sequence of glutathione transport enzyme (YCF1)
SEQ ID NO: 12: nucleotide sequence of nucleic acid encoding glutathione transport enzyme (YCF1)
SEQ ID NO: 13: forward PCR primer for ERV1 gene amplification
SEQ ID NO: 14: reverse PCR primer for ERV1 gene amplification
SEQ ID NO: 15: forward PCR primer for ERO1 gene amplification
SEQ ID NO: 16: reverse PCR primer for ERO1 gene amplification
SEQ ID NO: 17: forward PCR primer for YCF1 gene amplification
SEQ ID NO: 18: reverse PCR primer for YCF1 gene amplification

This application is based on a patent application No. 2015-042909 filed in Japan (filing date: Mar. 4, 2015), the contents of which are incorporated in full herein.

1-14. (canceled)

15. A method of producing glutathione, comprising:
(1) culturing a yeast having a thiol oxidase activity which is increased as compared to the parent strain of said yeast, in a culture medium to obtain a cultured broth comprising glutathione; and
(2) recovering said glutathione from said cultured broth.

16. The method according to claim 15, wherein said thiol oxidase is selected from the group consisting of:
(a) a protein having an amino acid sequence shown by SEQ ID NO: 1 or 2,
(b) a protein having an amino acid sequence shown by SEQ ID NO: 1 or 2, wherein 1 or plural amino acids are deleted, substituted, inserted and/or added, and having a thiol oxidase activity,
(c) a protein having an amino acid sequence having 60% or more sequence identity to the amino acid sequence shown by SEQ ID NO: 1 or 2,
(d) a protein having an amino acid sequence encoded by a nucleotide sequence shown by SEQ ID NO: 3 or 4,
(e) a protein having an amino acid sequence encoded by a DNA that hybridizes with a DNA having a nucleotide sequence complementary to SEQ ID NO: 3 or 4 under stringent conditions, and
(f) a protein having an amino acid sequence encoded by a DNA having the nucleotide sequence shown by SEQ ID NO: 3 or 4 wherein 1 or plural nucleotides are substituted, deleted, inserted and/or added, and having a thiol oxidase activity.

17. The method according to claim 15, wherein said yeast has a glutathione reductase activity which is reduced as compared to the parent strain of said yeast.

18. The method according to claim 17, wherein said glutathione reductase is selected from the group consisting of:
(a) a protein having the amino acid sequence shown by SEQ ID NO: 5,
(b) a protein having the amino acid sequence shown by SEQ ID NO: 5, wherein 1 or plural amino acids are deleted, substituted, inserted and/or added, and having a glutathione reductase activity,
(c) a protein having an amino acid sequence having 60% or more sequence identity to the amino acid sequence shown by SEQ ID NO: 5,
(d) a protein having an amino acid sequence encoded by a nucleotide sequence shown by SEQ ID NO: 6,
(e) a protein having an amino acid sequence encoded by a DNA that hybridizes with a DNA having a nucleotide sequence complementary to SEQ ID NO: 6 under stringent conditions, and
(f) a protein having an amino acid sequence encoded by a DNA having the nucleotide sequence shown by SEQ ID NO: 6 wherein 1 or plural nucleotides are substituted, deleted, inserted and/or added, and having a glutathione reductase activity.

19. The method according to claim 15, wherein in said yeast the expression of γ-glutamylcysteine synthetase and/or glutathione synthetase is enhanced as compared to the parent strain of said yeast.

20. The method according to claim 19, wherein said γ-glutamylcysteine synthetase is selected from the group consisting of
(a) a protein having the amino acid sequence shown by SEQ ID NO: 7,
(b) a protein having the amino acid sequence shown by SEQ ID NO: 7, wherein 1 or plural amino acids are deleted, substituted, inserted and/or added, and having a γ-glutamylcysteine synthetase activity,
(c) a protein having an amino acid sequence having 60% or more sequence identity to the amino acid sequence shown by SEQ ID NO: 7,
(d) a protein having an amino acid sequence encoded by a nucleotide sequence shown by SEQ ID NO: 8,
(e) a protein having an amino acid sequence encoded by a DNA that hybridizes with a DNA having a nucleotide sequence complementary to SEQ ID NO: 8 under stringent conditions, and
(f) a protein having an amino acid sequence encoded by a DNA having the nucleotide sequence shown by SEQ ID NO: 8 wherein 1 or plural nucleotides are substituted, deleted, inserted and/or added, and having a γ-glutamylcysteine synthetase activity.

21. The method according to claim 19, wherein said glutathione synthetase is selected from the group consisting of
(a) a protein having amino acid sequence shown by SEQ ID NO: 9,
(b) a protein having the amino acid sequence shown by SEQ ID NO: 9, wherein 1 or plural amino acids are deleted, substituted, inserted and/or added, and having a glutathione synthetase activity,
(c) a protein having an amino acid sequence having 60% or more sequence identity to the amino acid sequence shown by SEQ ID NO: 9,
(d) a protein having an amino acid sequence encoded by a nucleotide sequence shown by SEQ ID NO: 10,
(e) a protein having an amino acid sequence encoded by a DNA that hybridizes with a DNA having a nucleotide sequence complementary to SEQ ID NO: 10 under stringent conditions, and
(f) a protein having an amino acid sequence encoded by a DNA having the nucleotide sequence shown by SEQ ID NO: 10 wherein 1 or plural nucleotides are substituted, deleted, inserted and/or added, and having a glutathione synthetase activity.

22. The method according to claim 15, wherein in said yeast the expression of glutathione transport enzyme is enhanced as compared to the parent strain of said yeast.

23. The method according to claim 22, wherein said glutathione transport enzyme is selected from the group consisting of:
(a) a protein having the amino acid sequence shown by SEQ ID NO: 11,
(b) a protein having the amino acid sequence shown by SEQ ID NO: I I, wherein 1 or plural amino acids are deleted, substituted, inserted and/or added, and having a glutathione transport enzyme activity,
(c) a protein having an amino acid sequence having 60% or more sequence identity to the amino acid sequence shown by SEQ ID NO: 11,
(d) a protein having an amino acid sequence encoded by a nucleotide sequence shown by SEQ ID NO: 12,
(e) a protein having an amino acid sequence encoded by a DNA that hybridizes with a DNA having a nucleotide sequence complementary to SEQ ID NO: 12 under stringent conditions, and
(f) a protein having an amino acid sequence encoded by a DNA having the nucleotide sequence shown by SEQ ID NO: 12 wherein 1 or plural nucleotides are substituted, deleted, inserted and/or added, and having a glutathione transport enzyme activity.

24. The method according to claim 15, wherein said yeast belongs to the genus Saccharomyces, the genus Candida; or the genus Pichia.

25. A method of producing reduced glutathione, comprising:
(3) reducing oxidized glutathione in said glutathione obtained by a method according to claim 15.

26. A yeast having artificially modified genes such that the expression of thiol oxidase is enhanced and the expression of γ-glutamylcysteine synthetase and/or glutathione synthetase is enhanced as compared to the parent strain of said yeast.

27. A yeast having artificially modified genes such that the expression of thiol oxidase is enhanced and the expression of glutathione transport enzyme is enhanced as compared to the parent strain of said yeast.

28. The yeast according to claim 26, wherein said yeast belongs to the genus Saccharomyces, the genus Candida, or the genus Pichia.

29. The yeast according to claim 27, wherein said yeast belongs to the genus Saccharomyces, the genus Candida, or the genus Pichia.

30. The yeast according to claim 26, which expresses a thiol oxidase selected from the group consisting of:
(a) a protein having an amino acid sequence shown by SEQ ID NO: 1 or 2,
(b) a protein having an amino acid sequence shown by SEQ ID NO: 1 or 2, wherein 1 or plural amino acids are deleted, substituted, inserted and/or added, and having a thiol oxidase activity,
(c) a protein having an amino acid sequence having 60% or more sequence identity to the amino acid sequence shown by SEQ ID NO: 1 or 2,
(d) a protein having an amino acid sequence encoded by a nucleotide sequence shown by SEQ ID NO: 3 or 4,
(e) a protein having an amino acid sequence encoded by a DNA that hybridizes with a DNA having a nucleotide sequence complementary to SEQ ID NO: 3 or 4 under stringent conditions, and
(f) a protein having an amino acid sequence encoded by a DNA having the nucleotide sequence shown by SEQ ID NO: 3 or 4 wherein 1 or plural nucleotides are substituted, deleted, inserted and/or added, and having a thiol oxidase activity.

31. The yeast according to claim 26, which expresses a γ-glutamylcysteine synthetase selected from the group consisting of:
(a) a protein having the amino acid sequence shown by SEQ ID NO: 7,
(b) a protein having the amino acid sequence shown by SEQ ID NO: 7, wherein 1 or plural amino acids are deleted, substituted, inserted and/or added, and having a γ-glutamylcysteine synthetase activity,
(c) a protein having an amino acid sequence having 60% or more sequence identity to the amino acid sequence shown by SEQ ID NO: 7,
(d) a protein having an amino acid sequence encoded by a nucleotide sequence shown by SEQ ID NO: 8,
(e) a protein having an amino acid sequence encoded by a DNA that hybridizes with a DNA having a nucleotide sequence complementary to SEQ ID NO: 8 under stringent conditions, and
(f) a protein having an amino acid sequence encoded by a DNA having the nucleotide sequence shown by SEQ ID NO: 8 wherein 1 or plural nucleotides are substituted, deleted, inserted and/or added, and having a γ-glutamylcysteine synthetase activity.

32. The yeast according to claim 26, which expresses a glutathione synthetase selected from the group consisting of:
(a) a protein having amino acid sequence shown by SEQ ID NO: 9,
(b) a protein having the amino acid sequence shown by SEQ ID NO: 9, wherein 1 or plural amino acids are deleted, substituted, inserted and/or added, and having a glutathione synthetase activity,
(c) a protein having an amino acid sequence having 60% or more sequence identity to the amino acid sequence shown by SEQ ID NO: 9,
(d) a protein having an amino acid sequence encoded by a nucleotide sequence shown by SEQ ID NO: 10,
(e) a protein having an amino acid sequence encoded by a DNA that hybridizes with a DNA having a nucleotide sequence complementary to SEQ ID NO: 10 under stringent conditions, and
(f) a protein having an amino acid sequence encoded by a DNA having the nucleotide sequence shown by SEQ ID NO: 10 wherein 1 or plural nucleotides are substituted, deleted, inserted and/or added, and having a glutathione synthetase activity.

33. The yeast according to claim 27, which expresses a thiol oxidase selected from the group consisting of:
(a) a protein having an amino acid sequence shown by SEQ ID NO: 1 or 2,
(b) a protein having an amino acid sequence shown by SEQ ID NO: 1 or 2, wherein 1 or plural amino acids are deleted, substituted, inserted and/or added, and having a thiol oxidase activity,
(c) a protein having an amino acid sequence having 60% or more sequence identity to the amino acid sequence shown by SEQ ID NO: 1 or 2,
(d) a protein having an amino acid sequence encoded by a nucleotide sequence shown by SEQ ID NO: 3 or 4,
(e) a protein having an amino acid sequence encoded by a DNA that hybridizes with a DNA having a nucleotide sequence complementary to SEQ ID NO: 3 or 4 under stringent conditions, and
(f) a protein having an amino acid sequence encoded by a DNA having the nucleotide sequence shown by SEQ ID NO: 3 or 4 wherein 1 or plural nucleotides are substituted, deleted, inserted and/or added, and having a thiol oxidase activity.

34. The yeast according to claim 27, which expresses a glutathione transport enzyme selected from the group consisting of:
(a) a protein having the amino acid sequence shown by SEQ ID NO: 11,
(b) a protein having the amino acid sequence shown by SEQ ID NO: 11, wherein 1 or plural amino acids are deleted, substituted, inserted and/or added, and having a glutathione transport enzyme activity,
(c) a protein having an amino acid sequence having 60% or more sequence identity to the amino acid sequence shown by SEQ ID NO: 11,
(d) a protein having an amino acid sequence encoded by a nucleotide sequence shown by SEQ ID NO: 12,
(e) a protein having an amino acid sequence encoded by a DNA that hybridizes with a DNA having a nucleotide sequence complementary to SEQ ID NO: 12 under stringent conditions, and
(f) a protein having an amino acid sequence encoded by a DNA having the nucleotide sequence shown by SEQ ID NO: 12 wherein 1 or plural nucleotides are substituted, deleted, inserted and/or added, and having a glutathione transport enzyme activity.
